(19) United States
(12) Patent Application Publication
(10) Pub. No.: US 2021/0095032 A1
(43) Pub. Date: Apr. 1, 2021

(54) ANTIBODIES BINDING PD-1 AND USES THEREOF

(71) Applicant: Salubris (Chengdu) Biotech Co., Ltd., Chengdu (CN)

(72) Inventors: John LI, Chengdu (CN); Mingjiu CHEN, Nanjing, Jiangsu Province (CN); Wei TAN, Nanjing, Jiangsu Province (CN); Yong TANG, Chengdu (CN); Shengwei LI, Chengdu (CN); Ming ZHOU, Chengdu (CN)

(21) Appl. No.: 17/043,809

(22) PCT Filed: Apr. 12, 2019
(86) PCT No.: PCT/CN2019/082447

§ 371 Date: Sep. 30, 2020

Related U.S. Application Data
(60) Provisional application No. 62/795,573, filed on Jan. 23, 2019.
(60) Provisional application No. 62/657,927, filed on Apr. 15, 2018.

(51) Int. Cl. C07K 16/28; A61K 39/395; A61K 45/06; A61P 35/00

(52) U.S. Cl. CPC C07K 16/2818; A61K 39/39541; A61K 2039/505; A61P 35/00; A61K 45/06

(57) ABSTRACT

An isolated monoclonal antibody or an antigen-binding fragment that specifically binds human PD-1. A nucleic acid molecule encoding the antibody or the antigen-binding fragment, an expression vector, a host cell and a method for expressing the antibody or the antigen-binding fragment are also provided. The present invention further provides an immunoconjugate, a bispecific molecule, a chimeric antigen receptor, an oncolytic virus and a pharmaceutical composition comprising the antibody or the antigen-binding fragment, as well as a treatment method using an anti-PD-1 antibody of the invention.

### REFERENCES TO RELATED APPLICATIONS

This application is a U.S. national stage of PCT/CN2019/082447 filed on Apr. 12, 2019, which claims the benefits of the U.S. patent application No. 62/657,927 filed on Apr. 15, 2018, and U.S. patent application No. 62/795,573 filed on Jan. 23, 2019, the contents of which are all incorporated herein by reference in their entireties.

### SEQUENCE LISTING

The instant application contains a Sequence Listing which has been submitted in ASCII format via EFS-Web and is hereby incorporated by reference in its entirety. Said ASCII copy, created on Apr. 11, 2019, is named 059541-070USPX-SL.txt and is 57,740 bytes in size.

### FIELD OF THE INVENTION

The present invention relates generally to an isolated monoclonal antibody, particularly a mouse, chimeric or humanized monoclonal antibody that specifically binds to human PD-1 with high affinity and functionality. A nucleic acid molecule encoding the antibody, an expression vector, a host cell and a method for expressing the antibody are also provided. The present invention further provides an immunoconjugate, a bispecific molecule and a pharmaceutical composition comprising the antibody, as well as a diagnostic and treatment method using an anti-PD-1 antibody of the invention.

### BACKGROUND OF THE INVENTION

Programmed cell death protein 1, also known as PD-1 or CD279, is a member of the CD28 family of T cell regulators, and expressed on activated B cells, T cells, and myeloid cells (Agata et al., (1996) Int Immunol 8:765-72; Okazaki et al., (2002) Curr. Opin. Immunol. 14: 391779-82; Bennett et al., (2003)J Immunol 170:711-8). It contains a membrane proximal immunoreceptor tyrosine inhibitory motif (ITIM) and a membrane distal tyrosine-based switch motif (ITSM) (Thomas, M. L. (1995) J Exp Med 181:1953-6; Vivier, E and Daeron, M (1997) Immunol Today 18:286-91). Two ligands for PD-1 have been identified, PD-L1 and PD-L2, both are B7 homologs that bind to PD-1, but do not bind to other CD28 family members.

Several lines of evidence have suggested that PD-1 and its ligands negatively regulate immune responses. For example, PD-1 was found abundant in a variety of human cancers (Dong et al., (2002) Nat. Med. 8:787-9). Further, the interaction between PD-1 and PD-L1 was reported to cause a decrease in tumor infiltrating lymphocytes as well as T-cell receptor mediated proliferation, and to induce immune evasion of cancerous cells (Dong et al., (2003) J. Mol. Med. 81:281-7; Blank et al., (2005) Cancer Immunol. Immunother. 54:307-314; Konishi et al., (2004) Clin. Cancer Res. 10:5094-100). Studies also showed that immune suppression can be reversed by inhibiting the local interaction of PD-1 with PD-L1, and the effect was accumulative when the interaction of PD-1 with PD-L2 was blocked as well (Iwai et al., (2002) Proc. Nat'l. Acad. Sci. USA 99:12293-7; Brown et al., (2003) J Immunol. 170:1257-66).

PD-1 deficient animals may develop various autoimmune phenotypes, including autoimmune cardiomyopathy and a lupus-like syndrome with arthritis and nephritis (Nishimura et al., (1999) Immunity 11:141-51; Nishimura et al., (2001) Science 291:319-22). Additionally, PD-1 has been found to play a role in autoimmune encephalomyelitis, systemic lupus erythematosus, graft-versus-host disease (GVHD), type I diabetes, and rheumatoid arthritis (Salama et al., (2003) J Exp Med 198:71-78; Prokunina and Alarcon-Riquelme (2004) Hum Mol Genet 13:R143; Nielsen et al., (2004) Lupus 13:510). In a murine B cell tumor line, the ITSM of PD-1 was shown to be essential to block BCR-mediated Ca²⁺-flux and tyrosine phosphorylation of downstream effector molecules (Okazaki et al., (2001) PNAS 98:13866-71).

A number of cancer immunotherapy agents that target the PD-1 receptor have been developed for disease treatment. One such anti-PD-1 antibody is Nivolumab (sold under the tradename of OPDIVO® by Bristol-Myers Squibb), which produced complete or partial responses in non-small-cell lung cancer, melanoma, and renal-cell cancer, in a clinical trial with a total of 296 patients (Topalian S L et al., (2012) The New England Journal of Medicine. 366 (26): 2443-54). It was approved in Japan in 2014 and by US FDA in 2014 to treat metastatic melanoma. Another anti-PD-1 antibody, Pembrolizumab (KEYTRUDA™, MK-3475, Merck & Co.) targeting PD-1 receptors, was also approved by US FDA in 2014 to treat metastatic melanoma. It is being used in clinical trials in US for lung cancer, lymphoma, and mesothelioma.

Despite the anti-PD-1 antibodies that are already developed and approved, there is a need for additional monoclonal antibodies with enhanced binding affinity to PD-1 and other desirable pharmaceutical characteristics.

### SUMMARY OF THE INVENTION

The present invention provides an isolated monoclonal antibody, for example, a mouse, human, chimeric or humanized monoclonal antibody, that binds to PD-1 (e.g., the human PD-1, and monkey PD-1) and has increased affinity to PD-1 and comparable, if not better, anti-tumor effect compared to existing anti-PD-1 antibodies such as Nivolumab.

The antibody of the invention can be used for a variety of applications, including detection of the PD-1 protein, and treatment and prevention of PD-1 associated diseases, such as cancers, autoimmune cardiomyopathy, autoimmune encephalomyelitis, systemic lupus erythematosus, graft-versus-host disease (GVHD), type I diabetes, and rheumatoid arthritis.

Accordingly, in one aspect, the invention pertains to an isolated monoclonal antibody (e.g., a mouse, chimeric or humanized antibody), or an antigen-binding portion thereof, that binds PD-1, having a heavy chain variable region that comprises a CDR1 region, a CDR2 region and a CDR3 region. The CDR1 region, the CDR2 region and the CDR3 region, when defined by IMGT numbering scheme, comprise amino acid sequences having at least 80%, 85%, 90%, 91%, 92%, 93%, 94%, 95%, 96%, 97%, 98%, 99% or 100% identity to (1) SEQ ID NOs: 1, 2 and 3, respectively; or (2) SEQ ID NOs: 4, 5 and 6, respectively, wherein, the antibody, or antigen-binding fragment thereof, binds to PD-1. The CDR1 region, the CDR2 region and the CDR3 region, when defined by Chothia numbering scheme, comprise amino acid sequences having at least 80%, 85%, 90%, 91%, 92%, 93%, 94%, 95%, 96%, 97%, 98%, 99% or 100% identity to (1) SEQ ID NOs: 37, 39 and 41, respectively; or (2) SEQ ID NOs: 44, 46 and 48, respectively, wherein, the antibody, or antigen-binding fragment thereof, binds to PD-1. The CDR1 region, the CDR2 region and the CDR3 region, when defined by Kabat numbering scheme, comprise amino acid sequences having at least 80%, 85%, 90%, 91%, 92%, 93%, 94%, 95%, 96%, 97%, 98%, 99% or 100% identity to (1) SEQ ID NOs: 38, 40 and 41, respectively; or (2) SEQ ID NOs: 45, 47 and 48, respectively, wherein, the antibody, or antigen-binding fragment thereof, binds to PD-1.

In one aspect, an isolated monoclonal antibody, or an antigen-binding portion thereof, of the present invention comprises a heavy chain variable region comprising an amino acid sequence having at least 80%, 85%, 90%, 91%, 92%, 93%, 94%, 95%, 96%, 97%, 98%, 99% or 100% identity to SEQ ID NOs: 13, 14, 15, 16, 17, 18, 19, 20, 21, 22, 23, 24, 25, or 26, wherein the antibody or antigen-binding fragment thereof binds to PD-1. SEQ ID NOs: 13, 21, 22 and 26 can be encoded by nucleic acid sequences of SEQ ID NOs: 55, 56, 57 and 58, respectively.

In one aspect, an isolated monoclonal antibody, or an antigen-binding portion thereof, of the present invention comprises a light chain variable region that comprises a CDR1 region, a CDR2 region and a CDR3 region. The CDR1 region, the CDR2 region, and the CDR3 region, when defined by Kabat numbering scheme or Chothia numbering scheme, comprise amino acid sequences having at least 80%, 85%, 90%, 91%, 92%, 93%, 94%, 95%, 96%, 97%, 98%, 99% or 100% identity to (1) SEQ ID NOs: 7, 8 and 9, respectively; or (2) SEQ ID NOs: 10, 11 and 12, respectively, wherein, the antibody, or antigen-binding fragment thereof, binds to PD-1. The CDR1 region, the CDR2 region, and the CDR3 region, when defined by IMGT numbering scheme, comprise amino acid sequences having at least 80%, 85%, 90%, 91%, 92%, 93%, 94%, 95%, 96%, 97%, 98%, 99% or 100% identity to (1) SEQ ID NOs: 42, 43 and 9, respectively; or (2) SEQ ID NOs: 49, 50 and 12, respectively, wherein, the antibody, or antigen-binding fragment thereof, binds to PD-1.

In one aspect, an isolated monoclonal antibody, or an antigen-binding portion thereof, of the present invention comprises a light chain variable region comprising an amino acid sequence having at least 80%, 85%, 90%, 91%, 92%, 93%, 94%, 95%, 96%, 97%, 98%, 99% or 100% identity to SEQ ID NOs: 27, 28, 29, 30, 31, 32, 33, 34, 35, or 36, wherein the antibody or antigen-binding fragment thereof binds to PD-1. SEQ ID NOs: 27, 33, 34 and 36 can be encoded by nucleic acid sequences of SEQ ID NOs: 59, 60, 61 and 62, respectively.

In one aspect, an isolated monoclonal antibody, or an antigen-binding portion thereof, of the present invention comprises a heavy chain variable region and a light chain variable region each comprising a CDR1 region, a CDR2 region and a CDR3 region, wherein the heavy chain variable region CDR1, CDR2 and CDR3, and the light chain variable region CDR1, CDR2 and CDR3 comprise amino acid sequences having at least 80%, 85%, 90%, 91%, 92%, 93%, 94%, 95%, 96%, 97%, 98%, 99% or 100% identity to (1) SEQ ID NOs: 1, 2, 3, 7, 8 and 9, respectively; or (2) SEQ ID NOs: 4, 5, 6, 10, 11 and 12, respectively; or (3) SEQ ID NOs: 1, 2, 3, 42, 43 and 9, respectively; or (4) SEQ ID NOs: 4, 5, 6, 49, 50 and 12, respectively; or (5) SEQ ID NOs: 37, 39, 41, 7, 8 and 9, respectively; or (6) SEQ ID NOs: 44, 46, 48, 10, 11 and 12, respectively; or (7) SEQ ID NOs: 37, 39, 41, 42, 43 and 9, respectively; or (8) SEQ ID NOs: 44, 46, 48, 49, 50 and 12, respectively; or (9) SEQ ID NOs: 38, 40, 41, 7, 8 and 9, respectively; or (10) SEQ ID NOs: 45, 47, 48, 10, 11 and 12, respectively; or (11) SEQ ID NOs: 38, 40, 41, 42, 43 and 9, respectively; or (12) SEQ ID NOs: 45, 47, 48, 49, 50 and 12, respectively, wherein the antibody or antigen-binding fragment thereof binds to PD-1.

In one embodiment, an isolated monoclonal antibody, or the antigen-binding portion thereof, of the present invention comprises a heavy chain variable region and a light chain variable region, the heavy chain variable region and the light chain variable region comprising amino acid sequences having at least 80%, 85%, 90%, 91%, 92%, 93%, 94%, 95%, 96%, 97%, 98%, 99% or 100% identity to (1) SEQ ID NOs: 13 and 27, respectively; (2) SEQ ID NOs: 14 and 28, respectively; (3) SEQ ID NOs: 15 and 28, respectively; (4) SEQ ID NOs: 16 and 28, respectively; (5) SEQ ID NOs: 17 and 28, respectively; (6) SEQ ID NOs: 18 and 28, respectively; (7) SEQ ID NOs: 19 and 28, respectively; (8) SEQ ID NOs: 20 and 28, respectively; (9) SEQ ID NOs: 14 and 29, respectively; (10) SEQ ID NOs: 14 and 30 respectively; (11) SEQ ID NOs: 14 and 31, respectively; (12) SEQ ID NOs: 14 and 32, respectively; (13) SEQ ID NOs: 21 and 28, respectively; (14) SEQ ID NOs: 14 and 33, respectively; (15) SEQ ID NOs: 21 and 33, respectively; (16) SEQ ID NOs: 22 and 34, respectively; (17) SEQ ID NOs: 23 and 35, respectively; (18) SEQ ID NOs: 24 and 35, respectively; (19) SEQ ID NOs: 25 and 35, respectively; (20) SEQ ID NOs: 23 and 36, respectively; (21) SEQ ID NOs: 26 and 35, respectively; or (22) SEQ ID NOs: 26 and 36, respectively, wherein the antibody or antigen-binding fragment thereof binds to PD-1.

In one embodiment, an isolated monoclonal antibody, or the antigen-binding portion thereof, of the present invention comprises a heavy chain and a light chain, the heavy chain comprising a heavy chain variable region and a heavy chain constant region, the light chain comprising a light chain variable region and a light chain constant region, wherein, the heavy chain constant region comprises amino acid sequences having at least 80%, 85%, 90%, 91%, 92%, 93%, 94%, 95%, 96%, 97%, 98%, 99% or 100% identity to SEQ ID No: 51 or 65, and the light chain constant region comprises amino acid sequences having at least 80%, 85%, 90%, 95%, 98%, 99% or 100% identity to SEQ ID No: 52 or 66, and the heavy chain variable region and the light chain variable region comprise amino acid sequences described above, wherein the antibody or antigen-binding fragment thereof binds to PD-1. These amino acid sequences of SEQ ID Nos: 51, 52, 65 and 66 can be encoded by nucleic acid sequences of SEQ ID NOs: 63, 64, 67 and 68, respectively.

The antibody of the present invention in some embodiments comprises or consists of two heavy chains and two light chains, wherein each heavy chain comprises the heavy chain constant region, heavy chain variable region or CDR sequences mentioned above, and each light chain comprises the light chain constant region, light chain variable region or CDR sequences mentioned above, wherein the antibody binds to PD-1. The antibody of the invention can be a full-length antibody, for example, of an IgG1, IgG2, IgG4 isotype or Fc-engineered IgGs. The antibody of the present invention in other embodiments may be a single chain antibody, or antibody fragments, such as Fab or Fab′2 fragments.

The antibody, or antigen-binding portion thereof, of the present invention has higher binding affinity to human PD-1 than prior anti-PD-1 antibodies such as Nivolumab, binding to human PD-1 with a K_{D} of 0.3-4.0×10⁻⁹M or less and inhibiting the binding of PD-L1 to PD-1. The antibody or antigen-binding portion thereof of the invention also provides comparable, if not better, anti-tumor effect compared to existing anti-PD-1 antibodies such as Nivolumab.

Nucleic acid molecules encoding the antibodies, or antigen-binding portions thereof, of the invention are also encompassed by the invention, as well as expression vectors comprising such nucleic acids and host cells comprising such expression vectors. A method for preparing an anti-PD-1 antibody using the host cell comprising the expression vector is also provided, comprising steps of (i) expressing the antibody in the host cell and (ii) isolating the antibody from the host cell or its cell culture.

The invention also provides an immunoconjugate comprising an antibody of the invention, or antigen-binding portion thereof, linked to a therapeutic agent, such as a cytotoxin, cytotoxic drug, etc. The invention also provides a bispecific molecule comprising an antibody, or antigen-binding portion thereof, of the invention, linked to a second functional moiety (e.g., a second antibody, cytokine, etc) having a different binding specificity than said antibody, or antigen-binding portion thereof. In another aspect, the antibody or an antigen binding portions thereof of the present invention can be made into part of a chimeric antigen receptor (CAR). The antibody or an antigen binding portions thereof of the present invention can also be encoded by or used in conjunction with an oncolytic virus.

Compositions comprising an antibody, or antigen-binding portion thereof, or immunoconjugate, bispecific molecule, or CAR of the invention, and a pharmaceutically acceptable carrier, are also provided.

In yet another aspect, the invention provides a method of modulating an immune response in a subject, comprising administering to the subject the antibody, or antigen-binding portion thereof, so that the immune response in the subject is modulated. Preferably, the antibody of the invention enhances, stimulates or increases the immune response in the subject. In some embodiments, the method comprises administering a composition, a bispecific molecule, an immunoconjugate, a CAR-T cell, or an antibody-encoding or antibody-bearing oncolytic virus of the invention, or alternatively a nucleic acid molecule capable of expressing the same in the subject.

In a further aspect, the invention provides a method of inhibiting tumor growth in a subject, comprising administering to a subject a therapeutically effective amount of the antibody, or antigen-binding portion thereof, of the present invention. The tumor may be a solid or non-solid tumor, including, but not limited to, lymphoma, leukemia, multiple myeloma, melanoma, colon adenocarcinoma, pancreas cancer, colon cancer, gastric intestine cancer, prostate cancer, bladder cancer, kidney cancer, ovary cancer, cervix cancer, breast cancer, lung cancer, renal-cell cancer and nasopharynx cancer. In some embodiments, the method comprises administering a composition, a bispecific molecule, an immunoconjugate, a CAR-T cell, or an antibody-encoding or antibody-bearing oncolytic virus of the invention, or alternatively a nucleic acid molecule capable of expressing the same in the subject.

In another aspect, the invention provides a method of treating an infectious disease in a subject, comprising administering to a subject a therapeutically effective amount of the antibody, or antigen-binding portion thereof, of the present invention. In some embodiments, the method comprises administering a composition, a bispecific molecule, an immunoconjugate, a CAR-T cell, or an antibody-encoding or antibody-bearing oncolytic virus of the invention, or alternatively a nucleic acid molecule capable of expressing the same in the subject.

Still further, the invention provides a method of enhancing an immune response to an antigen in a subject, comprising administering to the subject: (i) the antigen; and (ii) the antibody, or antigen-binding portion thereof, so that an immune response to the antigen in the subject is enhanced. The antigen can be, for example, a tumor antigen, a viral antigen, a bacterial antigen or an antigen from a pathogen.

The antibodies of the invention can be used in combination with at least one additional agent such as an immunostimulatory antibody (e.g., an anti-PD-L1 antibody and/or an anti-CTLA-4 antibody), a cytokine (e.g., IL-2 and/or IL-21), or a costimulatory antibody (e.g., an anti-CD137 and/or anti-GITR antibody).

Other features and advantages of the instant disclosure will be apparent from the following detailed description and examples, which should not be construed as limiting. The contents of all references, Genbank entries, patents and published patent applications cited throughout this application are expressly incorporated herein by reference.

### BRIEF DESCRIPTION OF THE DRAWINGS

FIG. 1 shows mice body weight changes in groups administered with the humanized anti-PD-1 antibodies or control agents.

FIG. 2A-2C show tumor volumes of mice administered with the humanized anti-PD-1 antibodies or control agents at a dose of 1 mg/kg (A), 3 mg/kg (B) or 10 mg/kg (C).

### DETAILED DESCRIPTION OF THE INVENTION

To ensure that the present disclosure may be more readily understood, certain terms are first defined. Additional definitions are set forth throughout the detailed description.

The term “PD-1” refers to programmed cell death protein 1. The term “PD-1” comprises variants, isoforms, homologs, orthologs and paralogs. For example, an antibody specific for a human PD-1 protein may, in certain cases, cross-react with a PD-1 protein from a species other than human, such as monkey. In other embodiments, an antibody specific for a human PD-1 protein may be completely specific for the human PD-1 protein and exhibit no cross-reactivity to other species or of other types, or may cross-react with PD-1 from certain other species but not all other species.

The term “human PD-1” refers to a PD-1 protein having an amino acid sequence from a human, such as the amino acid sequence of human PD-1 having Genbank Accession No. NP_005009.2. The terms “monkey or rhesus PD-1” and “mouse PD-1” refer to monkey and mouse PD-1 sequences, respectively, e.g. those with the amino acid sequences having Genbank Accession Nos. NP_001107830 and CAA48113, respectively.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

An “antigen-specific T cell response” refers to responses by a T cell that result from stimulation of the T cell with the antigen for which the T cell is specific. Non-limiting examples of responses by a T cell upon antigen-specific stimulation include proliferation, cytokine production (e.g., IL-2 production), and killing of antigen-positive cells.

The term “antibody” as referred to herein includes whole antibodies and any antigen binding fragment (i.e., “antigen-binding portion”) or single chains thereof. Whole antibodies are glycoproteins comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., a PD-1 protein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (or nanobody) (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded by separate genes, they can be joined, using recombinant methods, by a synthetic linker that enables them to be made as a single polypeptide chain in which the V_{L} and V_{H} regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds a PD-1 protein is substantially free of antibodies that specifically bind antigens other than PD-1 proteins). An isolated antibody that specifically binds a human PD-1 protein may, however, have cross-reactivity to other antigens, such as PD-1 proteins from other species. Moreover, an isolated antibody can be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “mouse antibody”, as used herein, is intended to include antibodies having variable regions in which both the framework and CDR regions are derived from mouse germline immunoglobulin sequences. Furthermore, if the antibody contains a constant region, the constant region also is derived from mouse germline immunoglobulin sequences. The mouse antibodies of the invention can include amino acid residues not encoded by mouse germline immunoglobulin sequences (e.g., mutations introduced by random or site-specific mutagenesis in vitro or by somatic mutation in vivo). However, the term “mouse antibody”, as used herein, is not intended to include antibodies in which CDR sequences derived from the germline of another mammalian species have been grafted onto mouse framework sequences.

The term “chimeric antibody” refers to an antibody made by combining genetic material from a nonhuman source with genetic material from a human being. Or more generally, a chimetic antibody is an antibody having genetic material from a certain species with genetic material from another species.

The term “humanized antibody”, as used herein, refers to an antibody from non-human species whose protein sequences have been modified to increase similarity to antibody variants produced naturally in humans.

The term “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

As used herein, an antibody that “specifically binds to human PD-1” is intended to refer to an antibody that binds to human PD-1 protein (and possibly a PD-1 protein from one or more non-human species) but does not substantially bind to non-PD-1 proteins. Preferably, the antibody binds to human PD-1 protein with “high affinity”, namely with a K_{D} of 1.0×10⁻⁹ M or less, more preferably 3.0×10⁻¹⁰ M or less.

The term “does not substantially bind” to a protein or cells, as used herein, means does not bind or does not bind with a high affinity to the protein or cells, i.e. binds to the protein or cells with a K_{D} of 1.0×10⁻⁶ M or more, more preferably 1.0×10⁻⁵ M or more, more preferably 1.0×10⁻⁴ M or more, more preferably 1.0×10⁻³ M or more, even more preferably 1.0×10⁻² M or more.

The term “high affinity” for an IgG antibody refers to an antibody having a K_{D} of 1.0×10⁻⁶ M or less, more preferably 5.0×10⁻⁸ M or less, even more preferably 1.0×10⁻⁸ M or less, even more preferably 4.0×10⁻⁹ M or less and even more preferably 1.0×10⁻⁹ M or less for a target antigen. However, “high affinity” binding can vary for other antibody isotypes. For example, “high affinity” binding for an IgM isotype refers to an antibody having a K_{D} of 10⁻⁶ M or less, more preferably 10⁻⁷ M or less, even more preferably 10⁻⁸ M or less.

The term “K_{assoc}” or “Kₐ”, as used herein, is intended to refer to the association rate of a particular antibody-antigen interaction, whereas the term “K_{dis}” or “Kₐ”, as used herein, is intended to refer to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The term “EC₅₀”, also known as half maximal effective concentration, refers to the concentration of an antibody which induces a response halfway between the baseline and maximum after a specified exposure time.

The term “IC₅₀”, also known as half maximal inhibitory concentration, refers to the concentration of an antibody which inhibits a specific biological or biochemical function by 50% relative to the absence of the antibody.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

The term “therapeutically effective amount” means an amount of the antibody of the present invention sufficient to prevent or ameliorate the symptoms associated with a disease or condition (such as a cancer) and/or lessen the severity of the disease or condition. A therapeutically effective amount is understood to be in context to the condition being treated, where the actual effective amount is readily discerned by those of skill in the art.

Various aspects of the invention are described in further detail in the following subsections.

Anti-PD-1 Antibodies Having Increased Binding Affinity to Human PD-1 and Better Anti-Tumor Effect

The antibody, or the antigen-binding portion thereof, of the invention specifically binds to human PD-1 and have improved binding affinity as well as comparable, if not better, anti-tumor effect compared to previously described anti-PD-1 antibodies, particularly compared to Nivolumab.

The antibody, or the antigen-binding portion thereof, of the invention preferably binds to human PD-1 protein with a K_{D} of 1.0×10⁻⁹ M or less, more preferably with a K_{D} of 3.0×10⁻¹° M or less. The antibodies of the invention also bind to Cynomolgus monkey PD-1 with a K_{D} at about 1.0×10⁻⁸ M to 1.0×10⁻¹° M.

Additional functional properties include the capacity to block PD-1/PD-L1 interaction. The antibodies of the present invention, in one embodiment, can inhibit binding of PD-1 to PD-L1 at a similar concentration as Nivolumab.

Other functional properties include the ability of the antibody to stimulate an immune response, such as an antigen-specific T cell response. This can be tested, for example, by assessing the ability of the antibody to stimulate interleukin-2 (IL-2) production in an antigen-specific T cell response. In certain embodiments, the antibody binds to human PD-1 and stimulates an antigen-specific T cell response. In other embodiments, the antibody binds to human PD-1 but does not stimulate an antigen-specific T cell response. Other means for evaluating the capacity of the antibody to stimulate an immune response include testing its ability to inhibit tumor growth, such as in an in vivo tumor graft model or the ability to stimulate an autoimmune response, such as the ability to promote the development of an autoimmune disease in an autoimmune model, e.g., the ability to promote the development of diabetes in the NOD mouse model.

Preferred antibodies of the invention are human monoclonal antibodies. Additionally or alternatively, the antibodies can be, for example, chimeric or humanized monoclonal antibodies.

Monoclonal Anti-PD-1 Antibody

A preferred antibody of the invention is the monoclonal antibody structurally and chemically characterized as described below and in the following Examples. The V_{H} amino acid sequence of the anti-PD-1 antibody is set forth in SEQ ID NOs: 13, 14, 15, 16, 17, 18, 19, 20, 21, 22, 23, 24, 25 or 26. The V_{L} amino acid sequence of the anti-PD-1 antibody is shown in SEQ ID NOs: 27, 28, 29, 30, 31, 32, 33, 34, 35 or 36. The amino acid sequences of the heavy/light chain variable regions of the antibodies are summarized in Table 1 below, some clones sharing the same V_{H} or V_{L}. The heavy chain constant region for all clones may be human IgG1 heavy chain constant region having an amino acid sequence set forth in, e.g., SEQ ID NO: 51, and the light chain constant region for all clones may be human kappa constant region having an amino acid sequence set forth in, e.g., SEQ ID NO: 52.

As is well known in the art, the CDR regions of the antibodies can be determined by the Kabat numbering system (Kabat et al., (Sequences of proteins of Immunological Interest NIH, 1987), the Chothia numbering system (Al-Lazikani et al., (1997) JMB 273, 927-948), the contact definition method (MacCallum R. M et al., (1996), Journal of Molecular Biology, 262 (5), 732-745) or any other established method for numbering the residues in an antibody and determining CDRs. Other numbering conventions for CDR sequences available to a skilled person include “AbM” (University of Bath) and “contact” (University College London) methods.

The heavy chain variable region CDRs and the light chain variable region CDRs in Table 1 have been defined by the IMGT numbering scheme and Kabat numbering scheme, respectively. Specific CDR sequences defined by different systems are summarized in Table 2.

TABLE 1
Amino acid sequence ID numbers of heavy/light chain variable regions

| Clone/SEQ ID NO. | V_{H}-CDR1 | V_{H}-CDR2 | V_{H}-CDR3 | V_{H} | V_{L}-CDR1 | V_{L}-CDR2 | V_{L}-CDR3 | V_{L} |
|---|---|---|---|---|---|---|---|---|
| Mouse and chimeric D2H3 | 1 | 2 | 3 | 13 | 7 | 8 | 9 | 27 |
| huD2H3-V1 | 1 | 2 | 3 | 14 | 7 | 8 | 9 | 28 |
| huD2H3-V2 | 1 | 2 | 3 | 15 | 7 | 8 | 9 | 28 |
| huD2H3-V3 | 1 | 2 | 3 | 16 | 7 | 8 | 9 | 28 |
| huD2H3-V4 | 1 | 2 | 3 | 17 | 7 | 8 | 9 | 28 |
| huD2H3-V5 | 1 | 2 | 3 | 18 | 7 | 8 | 9 | 28 |
| huD2H3-V6 | 1 | 2 | 3 | 19 | 7 | 8 | 9 | 28 |
| huD2H3-V7 | 1 | 2 | 3 | 20 | 7 | 8 | 9 | 28 |
| huD2H3-V8 | 1 | 2 | 3 | 14 | 7 | 8 | 9 | 29 |
| huD2H3-V9 | 1 | 2 | 3 | 14 | 7 | 8 | 9 | 30 |
| huD2H3-V10 | 1 | 2 | 3 | 14 | 7 | 8 | 9 | 31 |
| huD2H3-V11 | 1 | 2 | 3 | 14 | 7 | 8 | 9 | 32 |
| huD2H3-V12 | 1 | 2 | 3 | 21 | 7 | 8 | 9 | 28 |
| huD2H3-V13 | 1 | 2 | 3 | 14 | 7 | 8 | 9 | 33 |
| huD2H3-V14 | 1 | 2 | 3 | 21 | 7 | 8 | 9 | 33 |
| Mouse and chimeric D2A4 | 4 | 5 | 6 | 22 | 10 | 11 | 12 | 34 |
| huD2A4-V1 | 4 | 5 | 6 | 23 | 10 | 11 | 12 | 35 |
| huD2A4-V2 | 4 | 5 | 6 | 24 | 10 | 11 | 12 | 35 |
| huD2A4-V3 | 4 | 5 | 6 | 25 | 10 | 11 | 12 | 35 |
| huD2A4-V4 | 4 | 5 | 6 | 23 | 10 | 11 | 12 | 36 |
| huD2A4-V5 | 4 | 5 | 6 | 26 | 10 | 11 | 12 | 35 |
| huD2A4-V6 | 4 | 5 | 6 | 26 | 10 | 11 | 12 | 36 |

TABLE 2
Amino acid sequences and the SEQ ID numbers of heavy/light chain variable region CDRs

| | Numbering | | | | | | |
|---|---|---|---|---|---|---|---|
| Antibody | scheme | V_{H}-CDR1 | V_{H}-CDR2 | V_{H}-CDR3 | V_{L}-CDR1 | V_{L}-CDR2 | V_{L}-CDR3 |
| Mouse, | IMGT | GYTFTNYW | IFPRNSET | TRNRYGLDY | ESVSLHGTRL | LGS | QQSIEDPWT |
| chimeric and | | (SEQ ID | (SEQ ID NO.: 2) | (SEQ ID NO.: 3) | (SEQ ID NO.: 42) | (SEQ ID | (SEQ ID |
| humanized | | NO.: 1) | | | | NO.: 43) | NO.: 9) |
| D2H3 | Chothia | GYTFTNY | FPRNSE | NRYGLDY | RASESVSLHGTRLMH | LGSNLES | QQSIEDPWT |
| antibodies | | (SEQ ID | (SEQ ID NO.: 39) | (SEQ ID NO.: 41) | (SEQ ID NO.: 7) | (SEQ ID | (SEQ ID |
| | | NO.: 37) | | | | NO.: 8) | NO.: 9) |
| | Kabat | NYWMH | AIFPRNSETNYNQKFKA | NRYGLDY | RASESVSLHGTRLMH | LGSNLES | QQSIEDPWT |
| | | (SEQ ID | (SEQ ID NO.: 40) | (SEQ ID NO.: 41) | (SEQ ID NO.: 7) | (SEQ ID | (SEQ ID |
| | | NO.: 38) | | | | NO.: 8) | NO.: 9) |
| Mouse, | IMGT | GFTFSSYT | ISGGGSNT | ARQAFYSNYWYFDV | QTIGTW | AAT | QQVSSIPWT |
| chimeric and | | (SEQ ID | (SEQ ID NO.: 5) | (SEQ ID NO.: 6) | (SEQ ID NO.: 49) | (SEQ ID | (SEQ ID |
| humanized | | NO.: 4) | | | | NO.: 50) | NO.: 12) |
| D2A4 | Chothia | GFTFSSY | SGGGSN | QAFYSNYWYFDV | LASQTIGTWLA | AATSLAD | QQVSSIPWT |
| antibodies | | (SEQ ID | (SEQ ID NO.: 46) | (SEQ ID NO.: 48) | (SEQ ID NO.: 10) | (SEQ ID | (SEQ ID |
| | | NO.: 44) | | | | NO.: 11) | NO.: 12) |
| | Kabat | SYTMS | TISGGGSNTYYPDSVKG | QAFYSNYWYFDV | LASQTIGTWLA | AATSLAD | QQVSSIPWT |
| | | (SEQ ID | (SEQ ID NO.: 47) | (SEQ ID NO.: 48) | (SEQ ID NO.: 10) | (SEQ ID | (SEQ ID |
| | | NO.: 45) | | | | NO.: 11) | NO.: 12) |

The V_{H} and V_{L} sequences (or CDR sequences) of other anti-PD-1 antibodies which bind to human PD-1 can be “mixed and matched” with the V_{H} and V_{L} sequences (or CDR sequences) of the anti-PD-1 antibody of the present invention. Preferably, when V_{H} and V_{L} chains (or the CDRs within such chains) are mixed and matched, a V_{H} sequence from a particular V_{H}/V_{L} pairing is replaced with a structurally similar V_{H} sequence. Likewise, preferably a V_{L} sequence from a particular V_{H}/V_{L} pairing is replaced with a structurally similar V_{L} sequence.

Accordingly, in one embodiment, an antibody of the invention, or an antigen binding portion thereof, comprises:

(a) a heavy chain variable region comprising an amino acid sequence listed above in Table 1; and
(b) a light chain variable region comprising an amino acid sequence listed above in Table 1, or the V_{L} of another anti-PD-1 antibody, wherein the antibody specifically binds human PD-1.

In another embodiment, an antibody of the invention, or an antigen binding portion thereof, comprises:

(a) the CDR1, CDR2, and CDR3 regions of the heavy chain variable region listed above in Table 1/Table 2; and
(b) the CDR1, CDR2, and CDR3 regions of the light chain variable region listed above in Table 1/Table 2 or the CDRs of another anti-PD-1 antibody, wherein the antibody specifically binds human PD-1.

In yet another embodiment, the antibody, or antigen binding portion thereof, includes the heavy chain variable CDR2 region of anti-PD-1 antibody combined with CDRs of other antibodies which bind human PD-1, e.g., CDR1 and/or CDR3 from the heavy chain variable region, and/or CDR1, CDR2, and/or CDR3 from the light chain variable region of a different anti-PD-1 antibody.

In addition, it is well known in the art that the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, e.g., Klimka et al., British J. of Cancer 83(2):252-260 (2000); Beiboer et al., J. Mol. Biol. 296:833-849 (2000); Rader et al., Proc. Natl. Acad. Sci. U.S.A. 95:8910-8915 (1998); Barbas et al., J. Am. Chem. Soc. 116:2161-2162 (1994); Barbas et al., Proc. Natl. Acad. Sci. U.S.A. 92:2529-2533 (1995); Ditzel et al., J. Immunol. 157:739-749 (1996); Berezov et al., BIAjournal 8: Scientific Review 8 (2001); Igarashi et al., J. Biochem (Tokyo) 117:452-7 (1995); Bourgeois et al., J. Virol 72:807-10 (1998); Levi et al., Proc. Natl. Acad. Sci. U.S.A. 90:4374-8 (1993); Polymenis and Stoller, J. Immunol. 152:5218-5329 (1994) and Xu and Davis, Immunity 13:37-45 (2000). See also, U.S. Pat. Nos. 6,951,646; 6,914,128; 6,090,382; 6,818,216; 6,156,313; 6,827,925; 5,833,943; 5,762,905 and 5,760,185. Each of these references is hereby incorporated by reference in its entirety.

Accordingly, in another embodiment, antibodies of the invention comprise the CDR2 of the heavy chain variable region of the anti-PD-1 antibody and at least the CDR3 of the heavy and/or light chain variable region of the anti-PD-1 antibody, or the CDR3 of the heavy and/or light chain variable region of another anti-PD-1 antibody, wherein the antibody is capable of specifically binding to human PD-1. These antibodies preferably (a) compete for binding with PD-1; (b) retain the functional characteristics; (c) bind to the same epitope; and/or (d) have a similar binding affinity as the anti-PD-1 antibody of the present invention. In yet another embodiment, the antibodies further may comprise the CDR2 of the light chain variable region of the anti-PD-1 antibody, or the CDR2 of the light chain variable region of another anti-PD-1 antibody, wherein the antibody is capable of specifically binding to human PD-1. In another embodiment, the antibodies of the invention may include the CDR1 of the heavy and/or light chain variable region of the anti-PD-1 antibody, or the CDR1 of the heavy and/or light chain variable region of another anti-PD-1 antibody, wherein the antibody is capable of specifically binding to human PD-1.

Conservative Modifications

In another embodiment, an antibody of the invention comprises a heavy and/or light chain variable region sequences of CDR1, CDR2 and CDR3 sequences which differ from those of the anti-PD-1 antibodies of the present invention by one or more conservative modifications. It is understood in the art that certain conservative sequence modification can be made which do not remove antigen binding. See, e.g., Brummell et al., (1993) Biochem 32:1180-8; de Wildt et al., (1997) Prot. Eng. 10:835-41; Komissarov et al., (1997) J Biol. Chem. 272:26864-26870; Hall et al., (1992) J Immunol. 149:1605-12; Kelley and O'Connell (1993) Biochem. 32:6862-35; Adib-Conquy et al., (1998) Int. Immunol. 10:341-6 and Beers et al., (2000) Clin. Can. Res. 6:2835-43.

Accordingly, in one embodiment, the antibody comprises a heavy chain variable region comprising CDR1, CDR2, and CDR3 sequences and/or a light chain variable region comprising CDR1, CDR2, and CDR3 sequences, wherein:

(a) the heavy chain variable region CDR1 sequence comprises a sequence listed in Table 1/Table 2 above, and/or conservative modifications thereof; and/or
(b) the heavy chain variable region CDR2 sequence comprises a sequence listed in Table 1/Table 2 above, and/or conservative modifications thereof; and/or
(c) the heavy chain variable region CDR3 sequence comprises a sequence listed in Table 1/Table 2 above, and conservative modifications thereof; and/or
(d) the light chain variable region CDR1, and/or CDR2, and/or CDR3 sequences comprise the sequence(s) listed in Table 1/Table 2 above; and/or conservative modifications thereof; and
(e) the antibody specifically binds human PD-1.

The antibody of the present invention possesses one or more of the following functional properties described above, such as high affinity binding to human PD-1, and the ability to induce ADCC or CDC against PD-1-expressing cells.

In various embodiments, the antibody can be, for example, a mouse, human, humanized or chimeric antibody.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., the functions set forth above) using the functional assays described herein.

Engineered and Modified Antibodies

Antibodies of the invention can be prepared using an antibody having one or more of the V_{H}/V_{L} sequences of the anti-PD-1 antibody of the present invention as starting material to engineer a modified antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

In certain embodiments, CDR grafting can be used to engineer variable regions of antibodies. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad. See also U.S.A. 86:10029-10033; U.S. Pat. Nos. 5,225,539; 5,530,101; 5,585,089; 5,693,762 and 6,180,370).

Accordingly, another embodiment of the invention pertains to an isolated monoclonal antibody, or antigen binding portion thereof, comprising a heavy chain variable region comprising CDR1, CDR2, and CDR3 sequences comprising the sequences of the present invention, as described above, and/or a light chain variable region comprising CDR1, CDR2, and CDR3 sequences comprising the sequences of the present invention, as described above. While these antibodies contain the V_{H} and V_{L} CDR sequences of the monoclonal antibody of the present invention, they can contain different framework sequences.

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat et al., (1991), cited supra; Tomlinson et al., (1992) J Mol. Biol. 227:776-798; and Cox et al., (1994) Eur. J. Immunol. 24:827-836; the contents of each of which are expressly incorporated herein by reference. As another example, the germline DNA sequences for human heavy and light chain variable region genes can be found in the Genbank database. For example, the following heavy chain germline sequences found in the HCo7 HuMAb mouse are available in the accompanying Genbank Accession Nos.: 1-69 (NG-0010109, NT-024637 & BC070333), 3-33 (NG-0010109 & NT-024637) and 3-7 (NG-0010109 & NT-024637). As another example, the following heavy chain germline sequences found in the HCo12 HuMAb mouse are available in the accompanying Genbank Accession Nos.: 1-69 (NG-0010109, NT-024637 & BC070333), 5-51 (NG-0010109 & NT-024637), 4-34 (NG-0010109 & NT-024637), 3-30.3 (CAJ556644) & 3-23 (AJ406678).

Antibody protein sequences are compared against a compiled protein sequence database using one of the sequence similarity searching methods called the Gapped BLAST (Altschul et al., (1997), supra), which is well known to those skilled in the art.

Preferred framework sequences for use in the antibodies of the invention are those that are structurally similar to the framework sequences used by antibodies of the invention. The V_{H} CDR1, CDR2, and CDR3 sequences can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

Another type of variable region modification is to mutate amino acid residues within the V_{H} and/or V_{L} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as known in the art. Preferably conservative modifications (as known in the art) are introduced. The mutations can be amino acid substitutions, additions or deletions, but are preferably substitutions. Moreover, typically no more than one, two, three, four or five residues within a CDR region are altered.

Accordingly, in another embodiment, the invention provides isolated anti-PD-1 monoclonal antibodies, or antigen binding portions thereof, comprising a heavy chain variable region comprising: (a) a V_{H} CDR1 region comprising the sequence of the present invention, or an amino acid sequence having one, two, three, four or five amino acid substitutions, deletions or additions; (b) a V_{H} CDR2 region comprising the sequence of the present invention, or an amino acid sequence having one, two, three, four or five amino acid substitutions, deletions or additions; (c) a V_{H} CDR3 region comprising the sequence of the present invention, or an amino acid sequence having one, two, three, four or five amino acid substitutions, deletions or additions; (d) a V_{L} CDR1 region comprising the sequence of the present invention, or an amino acid sequence having one, two, three, four or five amino acid substitutions, deletions or additions; (e) a V_{L} CDR2 region comprising the sequence of the present invention, or an amino acid sequence having one, two, three, four or five amino acid substitutions, deletions or additions; and (f) a V_{L} CDR3 region comprising the sequence of the present invention, or an amino acid sequence having one, two, three, four or five amino acid substitutions, deletions or additions.

Engineered antibodies of the invention include those in which modifications have been made to framework residues within V_{H} and/or V_{L}, e.g. to improve the properties of the antibody. Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043.

In addition, or as an alternative to modifications made within the framework or CDR regions, antibodies of the invention can be engineered to include modifications within the Fc region, typically to alter one or more functional properties of the antibody, such as serum half-life, complement fixation, Fc receptor binding, and/or antibody-dependent cellular cytotoxicity. Furthermore, an antibody of the invention can be chemically modified (e.g., one or more chemical moieties can be attached to the antibody) or be modified to alter its glycosylation, again to alter one or more functional properties of the antibody.

In one embodiment, the hinge region of Cm is modified in such that the number of cysteine residues in the hinge region is altered, e.g., increased or decreased. This approach is described further in U.S. Pat. No. 5,677,425. The number of cysteine residues in the hinge region of Cm is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the C_{H2}-C_{H3} domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In still another embodiment, the glycosylation of an antibody is modified. For example, an aglycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. See, e.g., U.S. Pat. Nos. 5,714,350 and 6,350,861.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (α(1,6)-fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see U.S. Patent Publication No. 20040110704 and Yamane-Ohnuki et al., (2004) Biotechnol Bioeng 87:614-22). As another example, EP 1,176,195 describes a cell line with a functionally disrupted FUT8 gene, which encodes a fucosyl transferase, such that antibodies expressed in such a cell line exhibit hypofucosylation by reducing or eliminating the α-1,6 bond-related enzyme. EP 1,176,195 also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 describes a variant CHO cell line, Lec13 cells, with reduced ability to attach fucose to Asn(297)-linked carbohydrates, also resulting in hypofucosylation of antibodies expressed in that host cell (see also Shields et al., (2002) J. Biol. Chem. 277:26733-26740). Antibodies with a modified glycosylation profile can also be produced in chicken eggs, as described in PCT Publication WO 06/089231. Alternatively, antibodies with a modified glycosylation profile can be produced in plant cells, such as Lemna. PCT Publication WO 99/54342 describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al., (1999) Nat. Biotech. 17:176-180). Alternatively, the fucose residues of the antibody can be cleaved off using a fucosidase enzyme; e.g., the fucosidase α-L-fucosidase removes fucosyl residues from antibodies (Tarentino et al., (1975) Biochem. 14:5516-23).

Another modification of the antibodies herein that is contemplated by this disclosure is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See, e.g., EPO 154 316 and EP 0 401 384.

Antibody's Physical Properties

Antibodies of the invention can be characterized by their various physical properties, to detect and/or differentiate different classes thereof.

For example, antibodies can contain one or more glycosylation sites in either the light or heavy chain variable region. Such glycosylation sites may result in increased immunogenicity of the antibody or an alteration of the pK of the antibody due to altered antigen binding (Marshall et al (1972) Annu Rev Biochem 41:673-702; Gala and Morrison (2004) J Immunol 172:5489-94; Wallick et al (1988) J Exp Med 168:1099-109; Spiro (2002) Glycobiology 12:43R-56R; Parekh et al (1985) Nature 316:452-7; Mimura et al., (2000) Mol Immunol 37:697-706). Glycosylation has been known to occur at motifs containing an N-X-S/T sequence. In some instances, it is preferred to have an anti-PD-1 antibody that does not contain variable region glycosylation. This can be achieved either by selecting antibodies that do not contain the glycosylation motif in the variable region or by mutating residues within the glycosylation region.

In a preferred embodiment, the antibodies do not contain asparagine isomerism sites. The deamidation of asparagine may occur on N-G or D-G sequences and result in the creation of an isoaspartic acid residue that introduces a kink into the polypeptide chain and decreases its stability (isoaspartic acid effect).

Each antibody will have a unique isoelectric point (pI), which generally falls in the pH range between 6 and 9.5. The pI for an IgG1 antibody typically falls within the pH range of 7-9.5 and the pI for an IgG4 antibody typically falls within the pH range of 6-8. There is speculation that antibodies with a pI outside the normal range may have some unfolding and instability under in vivo conditions. Thus, it is preferred to have an anti-PD-1 antibody that contains a pI value that falls in the normal range. This can be achieved either by selecting antibodies with a pI in the normal range or by mutating charged surface residues.

Nucleic Acid Molecules Encoding Antibodies of the Invention

In another aspect, the invention provides nucleic acid molecules that encode heavy and/or light chain variable regions, or CDRs, of the antibodies of the invention. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques. A nucleic acid of the invention can be, e.g., DNA or RNA and may or may not contain intronic sequences. In a preferred embodiment, the nucleic acid is a cDNA molecule.

Nucleic acids of the invention can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), a nucleic acid encoding such antibodies can be recovered from the gene library.

Preferred nucleic acids molecules of the invention include those encoding the V_{H} and V_{L} sequences of the PD-1 monoclonal antibody or the CDRs. Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the V_{H} region can be converted to a full-length heavy chain gene by operatively linking the V_{H}-encoding DNA to another DNA molecule encoding heavy chain constant regions (C_{H1}, C_{H2} and C_{H3}). The sequences of human heavy chain constant region genes are known in the art and DNA fragments encompassing these regions can be obtained by standard PCR amplification. The heavy chain constant region can be an IgG1, IgG2, IgG3, IgG4, IgA, IgE, IgM or IgD constant region, but most preferably is an IgG1 or IgG4 constant region. For a Fab fragment heavy chain gene, the V_{H}-encoding DNA can be operatively linked to another DNA molecule encoding only the heavy chain C_{H1} constant region.

The isolated DNA encoding the V_{L} region can be converted to a full-length light chain gene (as well as a Fab light chain gene) by operatively linking the V_{L}-encoding DNA to another DNA molecule encoding the light chain constant region, C_{L}. The sequences of human light chain constant region genes are known in the art and DNA fragments encompassing these regions can be obtained by standard PCR amplification. In preferred embodiments, the light chain constant region can be a kappa or lambda constant region.

To create a scFv gene, the V_{H}- and V_{L}-encoding DNA fragments are operatively linked to another fragment encoding a flexible linker, e.g., encoding the amino acid sequence (Gly4-Ser)3, such that the V_{H} and V_{L} sequences can be expressed as a contiguous single-chain protein, with the V_{L} and V_{H} regions joined by the flexible linker (see e.g., Bird et al., (1988) Science 242:423-426; Huston et al., (1988) Proc. Natl. Acad. Sci. USA 85:5879-5883; McCafferty et al., (1990) Nature 348:552-554).

Production of Monoclonal Antibodies of the Invention

Monoclonal antibodies (mAbs) of the present invention can be produced using the well-known somatic cell hybridization (hybridoma) technique of Kohler and Milstein (1975) Nature 256: 495. Other embodiments for producing monoclonal antibodies include viral or oncogenic transformation of B lymphocytes and phage display techniques. Chimeric or humanized antibodies are also well known in the art. See e.g., U.S. Pat. Nos. 4,816,567; 5,225,539; 5,530,101; 5,585,089; 5,693,762 and 6,180,370, the contents of which are specifically incorporated herein by reference in their entirety.

Generation of Transfectomas Producing Monoclonal Antibodies of the Invention

Antibodies of the invention also can be produced in a host cell transfectoma using, for example, a combination of recombinant DNA techniques and gene transfection methods as is well known in the art (e.g., Morrison, S. (1985) Science 229:1202). In one embodiment, DNA encoding partial or full-length light and heavy chains obtained by standard molecular biology techniques is inserted into one or more expression vectors such that the genes are operatively linked to transcriptional and translational regulatory sequences. In this context, the term “operatively linked” is intended to mean that an antibody gene is ligated into a vector such that transcriptional and translational control sequences within the vector serve their intended function of regulating the transcription and translation of the antibody gene.

The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The antibody light chain gene and the antibody heavy chain gene can be inserted into the same or separate expression vectors. In preferred embodiments, the variable regions are used to create full-length antibody genes of any antibody isotype by inserting them into expression vectors already encoding heavy chain constant and light chain constant regions of the desired isotype such that the V_{H} segment is operatively linked to the CH segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes and regulatory sequences, the recombinant expression vectors of the invention can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody.

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982)J Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Immunoconjugates

Antibodies of the invention can be conjugated to a therapeutic agent to form an immunoconjugate such as an antibody-drug conjugate (ADC). Suitable therapeutic agents include cytotoxins, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In the ADC, the antibody and therapeutic agent preferably are conjugated via a linker cleavable such as a peptidyl, disulfide, or hydrazone linker. More preferably, the linker is a peptidyl linker such as Val-Cit, Ala-Val, Val-Ala-Val, Lys-Lys, Pro-Val-Gly-Val-Val, Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038,658; WO 07/051,081; WO 07/059,404; WO 08/083,312; and WO 08/103,693; U.S. Patent Publications 20060024317; 20060004081; and 20060247295; the disclosures of which are incorporated herein by reference.

Bispecific Molecules

In another aspect, the present disclosure features bispecific molecules comprising one or more antibodies of the invention linked to at least one other functional molecule, e.g., another peptide or protein (e.g., another antibody or ligand for a receptor) to generate a bispecific molecule that binds to at least two different binding sites or target molecules. Thus, as used herein, “bispecific molecule” includes molecules that have three or more specificities.

In an embodiment, a bispecific molecule has, in addition to an Fc binding specificity and an anti-PD-1 binding specificity, a third specificity. The third specificity can be for an anti-enhancement factor (EF), e.g., a molecule that binds to a surface protein involved in cytotoxic activity and thereby increases the immune response against the target cell. For example, the anti-enhancement factor can bind a cytotoxic T-cell (e.g. via CD2, CD3, CD8, CD28, CD4, PD-1, or ICAM-1) or other immune cell, resulting in an increased immune response against the target cell.

Bispecific molecules may be in many different formats and sizes. At one end of the size spectrum, a bispecific molecule retains the traditional antibody format, except that, instead of having two binding arms of identical specificity, it has two binding arms each having a different specificity. At the other extreme are bispecific molecules consisting of two single-chain antibody fragments (scFv's) linked by a peptide chain, a so-called Bs(scFv) 2 construct. Intermediate-sized bispecific molecules include two different F(ab) fragments linked by a peptidyl linker. Bispecific molecules of these and other formats can be prepared by genetic engineering, somatic hybridization, or chemical methods. See, e.g., Kufer et al, cited supra; Cao and Suresh, Bioconjugate Chemistry, 9 (6), 635-644 (1998); and van Spriel et al., Immunology Today, 21 (8), 391-397 (2000), and the references cited therein.

Antibody-Encoding or Antibody-Bearing Oncolytic Virus

An oncolytic virus preferentially infects and kills cancer cells. Antibodies of the present invention can be used in conjunction with oncolytic viruses. Alternatively, oncolytic viruses encoding antibodies of the present invention can be introduced into human body.

Pharmaceutical Compositions

In another aspect, the present disclosure provides a pharmaceutical composition comprising one or more antibodies of the present invention formulated together with a pharmaceutically acceptable carrier. The composition may optionally contain one or more additional pharmaceutically active ingredients, such as another antibody or a drug, such as an anti-tumor drug.

The pharmaceutical composition can comprise any number of excipients. Excipients that can be used include carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, an antibody of the invention can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a microemulsion, liposome, or other ordered structure suitable to high drug concentration.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about ninety-nine percent of active ingredient, preferably from about 0.1% to about 70%, most preferably from about 1% to about 30% of active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active ingredient calculated to produce the desired therapeutic effect in association with the required pharmaceutical carrier. Alternatively, antibody can be administered as a sustained release formulation, in which case less frequent administration is required.

For administration of the antibody, the dosage may range from about 0.0001 to 100 mg/kg, and more usually 0.01 to 10 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens for an anti-PD-1 antibody of the invention include 1-10 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/ml and in some methods about 25-300 μg/ml.

A “therapeutically effective dosage” of an anti-PD-1 antibody of the invention preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably inhibits tumor growth by at least about 20%, more preferably by at least about 40%, even more preferably by at least about 60%, and still more preferably by at least about 80% relative to untreated subjects. A therapeutically effective amount of a therapeutic antibody can decrease tumor size, or otherwise ameliorate symptoms in a subject, which is typically a human or can be another mammal.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

In certain embodiments, the monoclonal antibodies of the invention can be formulated to ensure proper distribution in vivo. For example, to ensure that the therapeutic antibody of the invention cross the blood-brain barrier, they can be formulated in liposomes, which may additionally comprise targeting moieties to enhance selective transport to specific cells or organs. See, e.g. U.S. Pat. Nos. 4,522,811; 5,374,548; 5,416,016; and 5,399,331; V. V. Ranade (1989) J. Clin. Pharmacol. 29:685; Umezawa et al., (1988) Biochem. Biophys. Res. Commun. 153:1038; Bloeman et al., (1995) FEBS Lett. 357:140; M. Owais et al., (1995) Antimicrob. Agents Chemother. 39:180; Briscoe et al., (1995)Am. J. Physiol. 1233:134; Schreier et al., (1994) J. Biol. Chem. 269:9090; Keinanen and Laukkanen (1994) FEBS Lett 346:123; and Killion and Fidler (1994) Immunomethods 4:273.

Uses and Methods of the Invention

Antibodies (compositions, bispecifics, and immunoconjugates) of the present invention have numerous in vitro and in vivo utilities involving, for example, enhancement of immune responses by blockade of PD-1. The antibodies can be administered to cells in culture, in vitro or ex vivo, or to human subjects, e.g., in vivo, to enhance immunity in a variety of situations. Accordingly, in one aspect, the invention provides a method of modifying an immune response in a subject comprising administering to the subject the antibody, or antigen-binding portion thereof, of the invention such that the immune response in the subject is modified. Preferably, the response is enhanced, stimulated or up-regulated.

Preferred subjects include human patients in need of enhancement of an immune response. The methods are particularly suitable for treating human patients having a disorder that can be treated by augmenting an immune response (e.g., the T-cell mediated immune response). In a particular embodiment, the methods are particularly suitable for treatment of cancer in vivo. To achieve antigen-specific enhancement of immunity, the anti-PD-1 antibodies can be administered together with an antigen of interest or the antigen may already be present in the subject to be treated (e.g., a tumor-bearing or virus-bearing subject). When antibodies to PD-1 are administered together with another agent, the two can be administered in either order or simultaneously.

Given the ability of anti-PD-1 antibodies of the invention to inhibit the binding of PD-1 to PD-L1 and/or PD-L2 molecules and to stimulate antigen-specific T cell responses, the invention also provides in vitro and in vivo methods of using the antibodies to stimulate, enhance or upregulate antigen-specific T cell responses. For example, the invention provides a method of stimulating an antigen-specific T cell response comprising contacting said T cell with an antibody of the invention, such that an antigen-specific T cell response is stimulated. Any suitable indicator of an antigen-specific T cell response can be used to measure the antigen-specific T cell response.

Non-limiting examples of such suitable indicators include increased T cell proliferation in the presence of the antibody and/or increase cytokine production in the presence of the antibody. In a preferred embodiment, interleukin-2 production by the antigen-specific T cell is stimulated.

The invention also provides method for stimulating an immune response (e.g., an antigen-specific T cell response) in a subject comprising administering an antibody of the invention to the subject such that an immune response (e.g., an antigen-specific T cell response) in the subject is stimulated. In a preferred embodiment, the subject is a tumor-bearing subject and an immune response against the tumor is stimulated. In another preferred embodiment, the subject is a virus-bearing subject and an immune response against the virus is stimulated.

In another embodiment, the invention provides methods for inhibiting growth of tumor cells in a subject comprising administering to the subject an antibody of the invention such that growth of the tumor is inhibited in the subject. In yet another embodiment, the invention provides methods for treating a viral infection in a subject comprising administering to the subject an antibody of the invention such that the viral infection is treated in the subject.

These and other methods of the invention are discussed in further detail below.

Cancer

Blockade of PD-1 by antibodies can enhance the immune response to cancerous cells in the patient. In one aspect, the present invention relates to treatment of a subject in vivo using an anti-PD-1 antibody such that growth of cancerous tumors is inhibited. An anti-PD-1 antibody can be used alone to inhibit the growth of cancerous tumors. Alternatively, an anti-PD-1 antibody can be used in conjunction with other immunogenic agents used in cancer treatments such as oncolytic viruses, or other antibodies, as described below.

Accordingly, in one embodiment, the invention provides a method of inhibiting growth of tumor cells or the prevention and/or treatment of cancer diseases in a subject, comprising administering to the subject a therapeutically effective amount of an anti-PD-1 antibody, or antigen-binding portion thereof. Preferably, the antibody is a mouse, chimeric or humanized anti-PD-1 antibody.

Preferred cancers whose growth may be inhibited using the antibodies of the invention include cancers typically responsive to immunotherapy. Non-limiting examples of preferred cancers for treatment include melanoma (e.g., metastatic malignant melanoma), renal cancer (e.g. clear cell carcinoma), prostate cancer (e.g. hormone refractory prostate adenocarcinoma), breast cancer, colon cancer and lung cancer (e.g. non-small cell lung cancer), whether original or metastatic. Additionally, the invention includes refractory or recurrent malignancies whose growth may be inhibited using the antibodies of the invention.

Examples of other cancers that can be treated using the methods of the invention include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The present invention is also useful for treatment of metastatic cancers, especially metastatic cancers that express PD-L1 (Iwai et al. (2005) Int. Immunol. 17:133-144).

Optionally, antibodies to PD-1 can be combined with an immunogenic agent, such as cancerous cells, purified tumor antigens (including recombinant proteins, peptides, and carbohydrate molecules), and cells transfected with genes encoding immune stimulating cytokines (He et al (2004) J. Immunol. 173:4919-28). Non-limiting examples of tumor vaccines that can be used include peptides of melanoma antigens, such as peptides of gp100, MAGE antigens, Trp-2, MARTI_ and/or tyrosinase, or tumor cells transfected to express the cytokine GM-CSF.

PD-1 blockade is likely to be more effective when combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci. U.S.A. 90: 3539-43).

The study of gene expression and large scale gene expression patterns in various tumors has led to the definition of so called tumor specific antigens (Rosenberg, S A (1999) Immunity 10: 281-7). In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. PD-1 blockade can be used in conjunction with a collection of recombinant proteins and/or peptides expressed in a tumor in order to generate an immune response to these proteins. These proteins are normally viewed by the immune system as self-antigens and are therefore tolerant to them. The tumor antigen can include the protein telomerase, which is required for the synthesis of telomeres of chromosomes and which is expressed in more than 85% of human cancers and in only a limited number of somatic tissues (Kim et al. (1994) Science 266: 2011-2013). These somatic tissues may be protected from immune attack by various means. Tumor antigen can also be “neo-antigens” expressed in cancer cells because of somatic mutations that alter protein sequence or create fusion proteins between two unrelated sequences (i.e., bcr-abl in the Philadelphia chromosome), or idiotype from B cell tumors.

Other tumor vaccines can include the proteins from viruses implicated in human cancers such as Human Papilloma Viruses (HPV), Hepatitis Viruses (HBV and HCV) and Kaposi's Herpes Sarcoma Virus (KHSV). Another form of tumor specific antigen which can be used in conjunction with PD-1 blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot & Srivastava (1995) Science 269:1585-1588; Tamura et al. (1997) Science 278:117-120).

Dendritic cells (DC) are potent antigen presenting cells that can be used to prime antigen-specific responses. DCs can be produced ex vivo and loaded with various protein and peptide antigens as well as tumor cell extracts (Nestle et al. (1998) Nature Medicine 4: 328-332). DCs can also be transduced by genetic means to express these tumor antigens as well. DCs have also been fused directly to tumor cells for the purposes of immunization (Kugler et al. (2000) Nature Medicine 6:332-336). As a method of vaccination, DC immunization can be effectively combined with PD-1 blockade to activate more potent anti-tumor responses.

PD-1 blockade can also be combined with standard cancer treatments. PD-1 blockade can be effectively combined with chemotherapeutic regimes. In these instances, it may be possible to reduce the dose of chemotherapeutic reagent administered (Mokyr et al. (1998) Cancer Research 58: 5301-5304). An example of such a combination is an anti-PD-1 antibody in combination with decarbazine for the treatment of melanoma. Another example of such a combination is an anti-PD-1 antibody in combination with interleukin-2 (IL-2) for the treatment of melanoma. The scientific rationale behind the combined use of PD-1 blockade and chemotherapy is that cell death, that is a consequence of the cytotoxic action of most chemotherapeutic compounds, should result in increased levels of tumor antigen in the antigen presentation pathway. Other combination therapies that may result in synergy with PD-1 blockade through cell death are radiation, surgery, and hormone deprivation. Each of these protocols creates a source of tumor antigen in the host. Angiogenesis inhibitors can also be combined with PD-1 blockade. Inhibition of angiogenesis leads to tumor cell death which may feed tumor antigen into host antigen presentation pathways.

PD-1 blocking antibodies can also be used in combination with bispecific antibodies that target Fcα or Fcγ receptor-expressing effectors cells to tumor cells (see, e.g., U.S. Pat. Nos. 5,922,845 and 5,837,243). Bispecific antibodies can be used to target two separate antigens. For example, anti-Fc receptor/anti-tumor antigen (e.g., Her-2/neu) bispecific antibodies have been used to target macrophages to sites of tumor. This targeting may more effectively activate tumor specific responses. The T cell arm of these responses would be augmented by the use of PD-1 blockade. Alternatively, antigen may be delivered directly to DCs by the use of bispecific antibodies which bind to tumor antigen and a dendritic cell specific cell surface marker.

Tumors evade host immune surveillance by a large variety of mechanisms. Many of these mechanisms may be overcome by the inactivation of proteins which are expressed by the tumors and which are immunosuppressive. These include among others TGF-β (Kehrl et al. (1986) J. Exp. Med. 163: 1037-1050), IL-10 (Howard & O'Garra (1992) Immunology Today 13: 198-200), and Fas ligand (Hahne et al. (1996) Science 274: 1363-1365). Antibodies to each of these entities can be used in combination with anti-PD-1 to counteract the effects of the immunosuppressive agent and favor tumor immune responses by the host.

Other antibodies which activate host immune responsiveness can be used in combination with anti-PD-1 antibody. These include molecules on the surface of dendritic cells which activate DC function and antigen presentation. Anti-CD40 antibodies are able to substitute effectively for T cell helper activity (Ridge et al. (1998) Nature 393: 474-478) and can be used in conjunction with PD-1 antibodies (Ito et al. (2000) Immunobiology 201 (5) 527-40). Activating antibodies to T cell costimulatory molecules such as CTLA-4 (e.g., U.S. Pat. No. 5,811,097), OX-40 (Weinberg et al. (2000) Immunol 164: 2160-2169), 4-IBB (Melero et al. (1997) Nature Medicine 3: 682-685 (1997), and ICOS (Hutloff et al. (1999) Nature 397: 262-266) may also provide for increased levels of T cell activation.

There are also several experimental treatment protocols that involve ex vivo activation and expansion of antigen specific T cells and adoptive transfer of these cells into recipients in order to stimulate antigen-specific T cells against tumor (Greenberg & Riddell (1999) Science 285: 546-51). These methods can also be used to activate T cell responses to infectious agents such as CMV. Ex vivo activation in the presence of anti-PD-1 antibodies can increase the frequency and activity of the adoptively transferred T cells.

Infectious Diseases

Other methods of the invention are used to treat patients that have been exposed to particular toxins or pathogens. Accordingly, another aspect of the invention provides a method of treating an infectious disease in a subject comprising administering to the subject an anti-PD-1 antibody, or antigen-binding portion thereof, such that the subject is treated for the infectious disease. Preferably, the antibody is a chimeric or humanized antibody.

Similar to its application to tumors as discussed above, antibody mediated PD-1 blockade can be used alone, or as an adjuvant, in combination with vaccines, to stimulate the immune response to pathogens, toxins, and self-antigens. Examples of pathogens for which this therapeutic approach can be particularly useful, include pathogens for which there is currently no effective vaccine, or pathogens for which conventional vaccines are less than completely effective. These include, but are not limited to HIV, Hepatitis (A, B, & C), Influenza, Herpes, Giardia, Malaria, Leishmania, Staphylococcus aureus, Pseudomonas aeruginosa. PD-1 blockade is particularly useful against established infections by agents such as HIV that present altered antigens over the course of the infections. These novel epitopes are recognized as foreign at the time of anti-human PD-1 administration, thus provoking a strong T cell response that is not dampened by negative signals through PD-1.

Some examples of pathogenic viruses causing infections treatable by methods of the invention include HIV, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Some examples of pathogenic bacteria causing infections treatable by methods of the invention include chlamydia, rickettsial bacteria, mycobacteria, staphylococci, streptococci, pneumonococci, meningococci and gonococci, klebsiella, proteus, serratia, pseudomonas, legionella, diphtheria, salmonella, bacilli, cholera, tetanus, botulism, anthrax, plague, leptospirosis, and Lymes disease bacteria.

Some examples of pathogenic fungi causing infections treatable by methods of the invention include Candida (albicans, krusei, glabrata, tropicalis, etc.), Cryptococcus neoformans, Aspergillus (fumigatus, niger, etc.), Genus Mucorales (mucor, absidia, rhizopus), Sporothrix schenkii, Blastomyces dermatitidis, Paracoccidioides brasiliensis, Coccidioides immitis and Histoplasma capsulatum.

Some examples of pathogenic parasites causing infections treatable by methods of the invention include Entamoeba histolytica, Balantidium coli, Naegleria_fowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondii, Nippostrongylus brasiliensis.

In all of the above methods, PD-1 blockade can be combined with other forms of immunotherapy such as cytokine treatment (e.g., interferons, GM-CSF, G-CSF, IL-2), or bispecific antibody therapy, which provides for enhanced presentation of tumor antigens (see, e.g., Holliger (1993) Proc. Natl. Acad. Sci. USA 90:6444-6448; Poljak (1994) Structure 2:1121-1123).

Autoimmune Reactions

Anti-PD-1 antibodies may provoke and amplify autoimmune responses. Indeed, induction of anti-tumor responses using tumor cell and peptide vaccines reveals that many anti-tumor responses involve anti-self reactivities (van Elsas et al. (2001) J Exp. Med. 194:481-489; Overwijk, et al. (1999) Proc. Natl. Acad. Sci. U.S.A. 96: 2982-2987; Hurwitz, (2000) supra; Rosenberg & White (1996) J. Immunother Emphasis Tumor Immunol 19 (1): 81-4). Therefore, it is possible to consider using anti-PD-1 blockade in conjunction with various self-proteins in order to devise vaccination protocols to efficiently generate immune responses against these self-proteins for disease treatment.

Other self-proteins can also be used as targets such as IgE for the treatment of allergy and asthma, and TNFα for rheumatoid arthritis. Finally, antibody responses to various hormones may be induced by the use of anti-PD-1 antibody. Neutralizing antibody responses to reproductive hormones can be used for contraception. Neutralizing antibody response to hormones and other soluble factors that are required for the growth of particular tumors can also be considered as possible vaccination targets.

Analogous methods as described above for the use of anti-PD-1 antibody can be used for induction of therapeutic autoimmune responses to treat patients having an inappropriate accumulation of other self-antigens, such as cytokines such as TNFα, and IgE.

Combination Therapy

In another aspect, the invention provides methods of combination therapy in which an anti-PD-1 antibody (or antigen-binding portion thereof) of the present invention is co-administered with one or more additional antibodies that are effective in stimulating immune responses to thereby further enhance, stimulate or upregulate immune responses in a subject. In one embodiment, the invention provides a method for stimulating an immune response in a subject comprising administering to the subject an anti-PD-1 antibody and one or more additional immune-stimulatory antibodies, such as an anti-LAG-3 antibody, an anti-PD-L1 antibody and/or an anti-CTLA-4 antibody, such that an immune response is stimulated in the subject, for example to inhibit tumor growth or to stimulate an anti-viral response. In another embodiment, the subject is administered an anti-PD-1 antibody and an anti-LAG-3 antibody. In still another embodiment, the subject is administered an anti-PD-1 antibody and an anti-PD-L1 antibody. In yet another embodiment, the subject is administered an anti-PD-1 antibody and an anti-CTLA-4 antibody. In another embodiment, the at least one additional immune-stimulatory antibody (e.g., anti-PD-1, anti-PD-L1 and/or anti-CTLA-4 antibody) is a human antibody. Alternatively, the at least one additional immune-stimulatory antibody can be, for example, a chimeric or humanized antibody (e.g., prepared from a mouse anti-LAG-3, anti-PD-L1 and/or anti-CTLA-4 antibody).

In another embodiment, the invention provides a method for treating a hyperproliferative disease (e.g., cancer), comprising administering a PD-1 antibody and a CTLA-4 antibody to a subject. In further embodiments, the anti-PD-1 antibody is administered at a subtherapeutic dose, the anti-CTLA-4 antibody is administered at a subtherapeutic dose, or both are administered at a subtherapeutic dose. In another embodiment, the present invention provides a method for altering an adverse event associated with treatment of a hyperproliferative disease with an immunostimulatory agent, comprising administering an anti-PD-1 antibody and a subtherapeutic dose of anti-CTLA-4 antibody to a subject. In certain embodiments, the subject is human. In other embodiments, the anti-CTLA-4 antibody is human sequence monoclonal antibody 10D1 (described in PCT Publication WO 01/14424) and the anti-PD-1 antibody is mouse sequence monoclonal antibody, such as anti-PD-1 antibody C1H5 described herein. Other anti-CTLA-4 antibodies encompassed by the methods of the present invention include, for example, those disclosed in: WO 98/42752; WO 00/37504; U.S. Pat. No. 6,207,156; Hurwitz et al. (1998) Proc. Natl. Acad. Sci. USA 95(17):10067-10071; Camacho et al. (2004) J Clin. Oncology 22(145): Abstract No. 2505 (antibody CP-675206); and Mokyr et al. (1998) Cancer Res. 58:5301-5304. In certain embodiments, the anti-CTLA-4 antibody binds to human CTLA-4 with a K_{D} of 5×10⁻⁸M or less, binds to human CTLA-4 with a K_{D} of 1×10⁻⁸ M or less, binds to human CTLA-4 with a K_{D} of 5×10⁻⁹ M or less, or binds to human CTLA-4 with a K_{D} of between 1×10⁻⁸ M and 1×10⁻¹⁹ M or less.

In another embodiment, the present invention provides a method for treating a hyperproliferative disease (e.g., cancer), comprising administering an anti-PD-1 antibody and an anti-LAG-3 antibody to a subject.

In another embodiment, the present invention provides a method for treating a hyperproliferative disease (e.g., cancer), comprising administering an anti-PD-1 antibody and an anti-PD-L1 antibody to a subject.

Blockade of PD-1 and one or more second target antigens such as CTLA-4 and/or LAG-3 and/or PD-L1 by antibodies can enhance the immune response to cancerous cells in the patient. Cancers whose growth may be inhibited using the antibodies of the instant disclosure include cancers typically responsive to immunotherapy. Representative examples of cancers for treatment with the combination therapy of the instant disclosure include those cancers specifically listed above in the discussion of monotherapy with anti-PD-1 antibodies.

In certain embodiments, the combination of therapeutic antibodies discussed herein can be administered concurrently as a single composition in a pharmaceutically acceptable carrier, or concurrently as separate compositions with each antibody in a pharmaceutically acceptable carrier. In another embodiment, the combination of therapeutic antibodies can be administered sequentially.

Furthermore, if more than one dose of the combination therapy is administered sequentially, the order of the sequential administration can be reversed or kept in the same order at each time point of administration, sequential administrations can be combined with concurrent administrations, or any combination thereof.

Tumors evade host immune surveillance by a large variety of mechanisms. Many of these mechanisms may be overcome by the inactivation of proteins, which are expressed by the tumors and which are immunosuppressive. These include, among others, TGF-β (Kehrl et al. (1986) J. Exp. Med. 163: 1037-1050), IL-10 (Howard & O'Garra (1992) Immunology Today 13: 198-200), and Fas ligand (Hahne et al. (1996) Science 274: 1363-1365). In another example, antibodies to each of these entities can be further combined with an anti-PD-1 and anti-CTLA-4 and/or anti-LAG-3 and/or anti-PD-L1 antibody combination to counteract the effects of immunosuppressive agents and favor anti-tumor immune responses by the host.

Other antibodies that can be used to activate host immune responsiveness can be further used in combination with an anti-PD-1 and anti-CTLA-4 and/or anti-LAG-3 and/or anti-PD-L1 antibody combination. These include molecules on the surface of dendritic cells that activate DC function and antigen presentation. Anti-CD40 antibodies (Ridge et al., supra) can be used in conjunction with an anti-PD-1 and anti-CTLA-4 and/or anti-LAG-3 and/or anti-PD-L1 combination (Ito et al., supra). Other activating antibodies to T cell costimulatory molecules (Weinberg et al., supra, Melero et al. supra, Hutloff et al., supra) may also provide for increased levels of T cell activation.

As discussed above, bone marrow transplantation is currently being used to treat a variety of tumors of hematopoietic origin. A combined PD-1 and CTLA-4 and/or LAG-3 and/or PD-L1 blockade can be used to increase the effectiveness of the donor engrafted tumor specific T cells.

Several experimental treatment protocols involve ex vivo activation and expansion of antigen specific T cells and adoptive transfer of these cells into recipients of antigen-specific T cells against tumor (Greenberg & Riddell, supra). These methods can also be used to activate T cell responses to infectious agents such as CMV. Ex vivo activation in the presence of anti-PD-1 and anti-CTLA-4 and/or anti-LAG-3 and/or anti-PD-L1 antibodies can be expected to increase the frequency and activity of the adoptively transferred T cells.

In certain embodiments, the present invention provides a method for altering an adverse event associated with treatment of a hyperproliferative disease (e.g., cancer) with an immunostimulatory agent, comprising administering an anti-PD-1 antibody and a subtherapeutic dose of anti-CTLA-4 and/or anti-LAG-3 and/or anti-PD-L1 antibody to a subject. For example, the methods of the present invention provide for a method of reducing the incidence of immunostimulatory therapeutic antibody-induced colitis or diarrhea by administering a non-absorbable steroid to the patient. Because any patient who will receive an immunostimulatory therapeutic antibody is at risk for developing colitis or diarrhea induced by such an antibody, this entire patient population is suitable for therapy according to the methods of the present invention. Although steroids have been administered to treat inflammatory bowel disease (IBD) and prevent exacerbations of IBD, they have not been used to prevent (decrease the incidence of) IBD in patients who have not been diagnosed with IBD. The significant side effects associated with steroids, even non-absorbable steroids, have discouraged prophylactic use.

In further embodiments, a combination PD-1 and CTLA-4 and/or LAG-3 and/or PD-L1 blockade (i.e., immunostimulatory therapeutic antibodies anti-PD-1 and anti-CTLA-4 and/or anti-LAG-3 antibodies and/or anti-PD-L1 antibodies) can be further combined with the use of any non-absorbable steroid. As used herein, a “non-absorbable steroid” is a glucocorticoid that exhibits extensive first pass metabolism such that, following metabolism in the liver, the bioavailability of the steroid is low, i.e., less than about 20%. In one embodiment of the invention, the non-absorbable steroid is budesonide. Budesonide is a locally-acting glucocorticosteroid, which is extensively metabolized, primarily by the liver, following oral administration. ENTOCORT EC™ (Astra-Zeneca) is a pH- and time-dependent oral formulation of budesonide developed to optimize drug delivery to the ileum and throughout the colon. ENTOCORT EC™ is approved in the U.S. for the treatment of mild to moderate Crohn's disease involving the ileum and/or ascending colon. The usual oral dosage of ENTOCORT EC™ for the treatment of Crohn's disease is 6 to 9 mg/day. ENTOCORT EC™ is released in the intestines before being absorbed and retained in the gut mucosa. Once it passes through the gut mucosa target tissue, ENTOCORT EC™ is extensively metabolized by the cytochrome P450 system in the liver to metabolites with negligible glucocorticoid activity. Therefore, the bioavailability is low (about 10%). The low bioavailability of budesonide results in an improved therapeutic ratio compared to other glucocorticoids with less extensive first-pass metabolism. Budesonide results in fewer adverse effects, including less hypothalamic-pituitary suppression, than systemically-acting corticosteroids. However, chronic administration of ENTOCORT EC™ can result in systemic glucocorticoid effects such as hypercorticism and adrenal suppression. See PDR 58th ed. 2004; 608-610.

In still further embodiments, a combination PD-1 and CTLA-4 and/or LAG-3 and/or PD-L1 blockade (i.e., immunostimulatory therapeutic antibodies anti-PD-1 and anti-CTLA-4 and/or anti-LAG-3 and/or anti-PD-L1 antibodies) in conjunction with a non-absorbable steroid can be further combined with a salicylate. Salicylates include 5-ASA agents such as, for example: sulfasalazine (AZULFIDINE™, Pharmacia & Upjohn); olsalazine (DIPENTUM™, Pharmacia & Upjohn); balsalazide (COLAZAL™, Salix Pharmaceuticals, Inc.); and mesalamine (ASACOL™, Procter & Gamble Pharmaceuticals; PENTASA™, Shire US; CANASA™, Axcan Scandipharm, Inc.; ROWASA™, Solvay).

In accordance with the methods of the present invention, a salicylate administration in combination with anti-PD-1 and anti-CTLA-4 and/or anti-LAG-3 and/or anti-PD-L1 antibodies and a non-absorbable steroid can include any overlapping or sequential administration of the salicylate and the non-absorbable steroid for the purpose of decreasing the incidence of colitis induced by the immunostimulatory antibodies. Thus, for example, methods for reducing the incidence of colitis induced by the immunostimulatory antibodies according to the present invention encompass administering a salicylate and a non-absorbable steroid concurrently or sequentially (e.g., a salicylate is administered 6 hours after a non-absorbable steroid), or any combination thereof. Further, according to the present invention, a salicylate and a non-absorbable steroid can be administered by the same route (e.g., both are administered orally) or by different routes (e.g., a salicylate is administered orally and a non-absorbable steroid is administered rectally), which may differ from the route(s) used to administer the anti-PD-1 and anti-CTLA-4 and/or anti-LAG-3 and/or anti-PD-L1 antibodies.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1 Generation of Mouse Anti-PD-1 Monoclonal Antibodies Using Hybridoma Technology

Immunization

Mice were immunized according to the method as described in E Harlow, D. Lane, Antibody: A Laboratory Manual, Cold Spring Harbor Laboratory Press, Cold Spring Harbor, N.Y., 1998. Recombinant human PD-1 protein with human IgG1 Fc tag at the C-terminus (Acro biosystems, #PD-1-H5257, containing extra-cellular domain, AA Leu 25-Gln 167) was used as the immunogen. Human PD-1-his protein (Sino biological, #10377-H08H) was used for determining anti-sera titer and for screening hybridomas secreting antigen-specific antibodies.

In specific, each animal was injected with 25 μg human PD1 Fc protein in complete Freud's adjuvant (Sigma, St. Louis, Mo., USA), and then boosted for 2 to 3 times by injection of 25 μg human PD1 Fc protein in noncomplete Freud's adjuvant (Sigma, St. Louis, Mo., USA) depending on the anti-sera titer. The anti-sera titer was measured by ELISA-based screening using recombinant human PD1-his protein. Briefly, diluted sera (60 μl) was added to each well and incubated at 37° C. for 40 minutes. Plates were then washed 4 times, HRP-goat anti-mouse-IgG (Jackson Immuno research, Cat #115-036-071) was used for detection, and binding ODs were observed at 450 nm. Animals with good titers were given a final boost by intraperitoneal injection before hybridoma fusion.

Hybridoma Fusion and Screening

Cells of murine myeloma cell line (SP2/0-Ag14, ATCC #CRL-1581) were cultured to reach the log phase stage right before fusion. Spleen cells from immunized mice were prepared sterilely and fused with myeloma cells according to the method as described in Kohler G, and Milstein C, “Continuous cultures of fused cells secreting antibody of predefined specificity,” Nature, 256: 495-497(1975). Fused “hybrid cells” were subsequently dispensed into 96-well plates in DMEM/20% FCS/HAT media. Surviving hybridoma colonies were observed under the microscope seven to ten days post fusion. After two weeks, the supernatant from each well was subjected to ELISA-based screening using recombinant human PD1-his protein. Briefly, ELISA plates were coated with 60 μl of human PD1-his (Sino biological, #10377-H08H, 2.0 μg/ml in PBS) overnight at 4° C. Plates were washed 4 times with PBST and blocked with 200 μl blocking buffer (5% non-fatty milk in PBST). Diluted hybridoma supernatant (60 μl) was added to each well and incubated at 37° C. for 40 minutes. Plates were then washed 4 times, HRP-goat anti-mouse-IgG (Jackson Immuno research, Cat #115-036-071) was used for detection, and binding ODs were observed at 450 nm. Positive hybridoma secreting antibody that binds to human PD1-his were then selected and transferred to 24-well plates. Hybridoma clones producing antibodies that showed high specific binding and PD1/PDL1 blocking activity were subcloned, and antibodies produced by the subclones were purified by protein A affinity chromatography. Briefly, Protein A sepharose column (from bestchrom (Shanghai) Biosciences, Cat #AA0273) was washed using PBS buffer in 5 to 10 column volumes. Cell supernatants were passed through the columns, and then the columns were washed using PBS buffer until the absorbance for protein reached the baseline. The columns were eluted with elution buffer (0.1 M Glycine-HCl, pH 2.7), and immediately collected into 1.5 ml tubes with neutralizing buffer (1 M Tris-HCl, pH 9.0). Fractions containing IgG were pooled and dialyzed in PBS overnight at 4° C.

### Example 2 Affinity Determination of Mouse Anti-PD-1 Monoclonal Antibodies Using BIACORE Surface Plasmon Resonance Technology

Anti-PD-1 mouse monoclonal antibodies (mAbs) produced by the hybridoma clones of Example 1 were characterized for affinities and binding kinetics by Biacore T200 system (GE healthcare, Pittsburgh, Pa., USA).

Briefly, goat anti-mouse IgG (GE healthcare, Cat #BR100839, Human Antibody Capture Kit) was covalently linked to a CMS chip (carboxy methyl dextran coated chip) via primary amines, using a standard amine coupling kit (GE healthcare, Pittsburgh, Pa., USA) provided by Biacore. Un-reacted moieties on the biosensor surface were blocked with ethanolamine. Then purified anti-PD-1 antibodies and Nivolumab (OPDIVO®) at the concentration of 66.7 nM were flowed onto the chip at a flow rate of 10 μL/min. Then, recombinant human PD-1-his (Sino biological, #10377-H08H) or cynomolgus monkey PD-1-his protein (Acro biosystems, #PD-1-05223) in HBS EP buffer (provided by Biacore) was flowed onto the chip at a flow rate of 30 μL/min. The antigen-antibody association kinetics was followed for 2 minutes and the dissociation kinetics was followed for 10 minutes. The association and dissociation curves were fit to a 1:1 Langmuir binding model using BIA evaluation software.

The kₐ, k_{d} and K_{D} values were determined and shown in Table 3 below.

TABLE 3
Biacore Kinetics of Mouse Anti-PD-1 Monoclonal Antibodies
Binding to Human or Cynomolgus Monkey PD-1

| | Kinetics on Biacore | | | | | |
|---|---|---|---|---|---|---|
| | Human PD-1 | | | Cynomolgus PD-1 | | |
| | Kₐ | K_{d} | K_{D} | Kₐ | K_{d} | K_{D} |
| Clone | (M⁻¹s⁻¹) | (s⁻¹) | (M) | (M⁻¹s⁻¹) | (s⁻¹) | (M) |
| D2H3 | 3.26E+05 | 1.22E−04 | 3.73E−10 | 2.11E+05 | 3.08E−04 | 1.46E−09 |
| D2A4 | 1.57E+05 | 4.01E−04 | 2.55E−09 | 1.21E+05 | 7.93E−04 | 6.57E−09 |
| OPDIVO ® | 4.33E+05 | 1.41E−03 | 3.25E−09 | / | / | / |

The antibodies of the present invention bound to human PD-1 with a similar or much lower K_{D} than Nivolumab, indicating comparable or higher affinity to human PD-1.

### Example 3 Binding Activity of Mouse Anti-PD-1 Monoclonal Antibodies

96-well micro plates were coated with 2 μg/ml goat anti-mouse IgG Fcγ fragment (Jackson Immuno Research, #115-006-071,100 μl/well) in PBS and incubated overnight at 4° C. Plates were washed 4 times with wash buffer (PBS+0.05% Tween-20, PBST) and then blocked with 200 μl/well blocking buffer (5% w/v non-fatty milk in PBST) for 2 hours at 37° C. Plates were washed again and incubated with 100 μl/well purified anti-PD-1 antibodies of Example 1 and Nivolumab (0.004-66.7 nM, 5-fold serial dilution in 2.5% non-fatty milk in PBST) for 40 minutes at 37° C., and then washed 4 times again. Plates containing captured anti-PD-1 antibodies were incubated with biotin-labeled human PD-1 Fc protein (SEQ ID NO: 53, 60 nM in 2.5% non-fatty milk in PBST, 100 μl/well) for 40 minutes at 37° C., washed 4 times, and incubated with streptavidin conjugated HRP (1:10000 dilution in PBST, Jackson Immuno Research, #016-030-084, 100 μl/well) for 40 minutes at 37° C. After a final wash, plates were incubated with 100 μl/well ELISA substrate TMB (Innoreagents). The reaction was stopped in 15 minutes at 25° C. with 50 μl/well 1M H₂SO₄, and the absorbance was read at 450 nm. Data were analyzed using Graphpad Prism software and EC₅₀ values were reported.

The results were summarized in Table 4 below.

TABLE 4
Binding Activity of anti-PD-1 antibodies to Human PD-1

| Clone | Capture ELISA (EC₅₀, nM) |
|---|---|
| D2H3 | 0.15 |
| D2A4 | 0.19 |
| OPDIVO ® | 0.21 |

The result indicated that the antibodies of the present invention bound to human PD-1 specifically, with slightly lower EC₅₀ values than Nivolumab.

### Example 4 Functional Blockage Assays Using ELISA and Report Assays

4.1 Ligand Blocking ELISA

The ability of anti-PD-1 antibodies of the present invention to block the PD-1-PD-L1 interaction was measured using a competitive ELISA assay. Briefly, human PD-L1-Fc proteins (SEQ ID NO: 54) were coated on 96-well micro plates at 2 μg/mL PBS and incubated overnight at 4° C. The next day, plates were washed with wash buffer (PBS+0.05% Tween-20, PBST), and blocked with 5% non-fatty milk in PBST for 2 hours at 37° C. Plates were then washed again using wash buffer.

Dilutions of the anti-PD-1 antibodies of the present invention or Nivolumab (starting at 100 nM with a four-fold serial dilution) in biotin labeled human PD-1-Fc (SEQ ID NO: 53, 10 nM in 2.5% non-fatty milk in PBST) were prepared and incubated at room temperature for 40 minutes, and then the antibodies/PD-1-Fc-biotin mixtures (100 μl/well) were added to PD-L1-coated plates. After incubation at 37° C. for 40 minutes, plates were washed for 4 times using wash buffer. Then 100 μl/well streptavidin conjugated HRP was added and incubated for 40 minutes at 37° C. to detect biotin-labeled human PD-1 bound to PD-L1. Plates were washed again using wash buffer. Finally, TMB was added and the reaction was stopped using 1M H₂SO₄, and the absorbance was read at 450 nm. Data were analyzed using Graphpad Prism software and IC₅₀ values were reported.

4.2 Benchmark Blocking ELISA

The ability of the anti-PD-1 antibodies of the present invention to block Benchmark (Nivolumab)-human PD-1 binding was measured using a competitive ELISA assay. Briefly, Nivolumab was coated on 96-well micro plates at 2 μg/mL in PBS and incubated overnight at 4° C. The next day, plates were washed with wash buffer, and blocked with 5% non-fatty milk in PBST for 2 hours at 37° C. While blocking, biotin labeled human PD-1 Fc (SEQ ID NO:53, 10 nM in 2.5% non-fatty milk in PBST) was mixed with each of the antibodies to test (137 pM-100 nM, 3-fold serial dilution) and incubated for 40 minutes at 25° C. After washing, the PD-1/antibody mixtures (100 μl/well) were added to plates coated with Nivolumab and incubated for 40 minutes at 37° C. Plates were washed again with wash buffer, and then 100 μl/well SA-HRP was added and incubated for 40 minutes at 37° C. to detect biotin-labeled human PD-1 bound to Opdivo®. Plates were finally washed using wash buffer. TMB was added and the reaction was stopped using 1M H₂SO₄, and the absorbance was read at 450 nm. Data were analyzed using Graphpad Prism software and IC₅₀ values were reported.

4.3 Cell-Based Functional Assays

The activity of antibodies to block cell membrane PD-1/PD-L1 interaction was evaluated by using a cell-based reporter assay. This assay consisted of two genetically engineered cell lines, PD-1 Effector Cell Line (Genscript, GS-J2/PD-1) stably expressing human PD-1 and a luciferase reporter driven by an NFAT response element (NFAT-RE), and PD-L1 Cell Line (Genscript, GS-C2/PD-L1, APC cells) stably expressing human PD-L1 and an engineered cell surface protein-antigenic peptide/major histocompatibility complex (MHC). When these two cell lines were co-cultured, the T-cell receptor (TCR)-mediated luciferase expression of PD-1 effector cell (via of the NFAT pathway) was inhibited by PD-1/PD-L1 interaction.

The cell-based functional assay was carried out as follows. Briefly, PD-L1 cells at the log phase stage were seeded into 384-well cell culture plates at the density of 5*10⁵/ml. The next day, dilution of anti-PD-1 antibodies of the present invention or Nivolumab (starting from 333.3 nM, 5-fold serial dilution) in assay buffer (RPMI 1640+1% FBS) were prepared. Meanwhile, the media of PD-L1 cells in 384-well plates were discarded, and then the dilutions of anti-PD-1 antibodies (20 μl/well) and PD-1 effector cells (at the density of 6.25*10⁵/ml, 20 μl/well) were added to 384-well cell culture plates. After co-cultured at 37° C. for six hours, the plates were removed from the incubator and the luminescence of each well was read according to the manufacturer's instructions with One-Glo Luciferase Assay system (Promega, #E6120). The dose-response curves were analyzed using Graphpad Prism software and EC₅₀ values were reported.

The results of the three assays were summarized in Table 5 below.

TABLE 5
Anti-PD-1 antibodies’ Capacity for Blocking PD-1-PD-L1 interaction

| | Functional Blockage Assays | | |
|---|---|---|---|
| | Competition ELISA (IC₅₀, nM) | | Reporter Activation (EC₅₀, nM) |
| | human PD-1/human | Opdivo ®/human | human PD-1 cell (NFAT-luc)/human |
| Clone | PD-Ll | PD-1 | PD-Ll cell |
| D2H3 | 0.19 | 0.76 | 3.018 |
| D2A4 | 0.17 | 2.22 | 8.102 |
| OPDIVO ® | 0.12 | 3.79 | 13.62 |

It can be seen that the antibodies of the present invention were capable of blocking human PD-1-human PD-L1 interaction, having similar or lower EC₅₀ or IC₅₀ values than Nivolumab.

The data also showed that the antibodies of the present invention were able to block human PD-1-Nivolumab interaction, indicating that they bound to the same or similar epitope as Nivolumab did.

### Example 5 Generation and Characterization of Chimeric Antibodies

The variable domains of the heavy and light chain of the anti-PD1 mouse mAbs D2H3 and D2A4 were cloned in frame to human IgG1 heavy-chain and human kappa light-chain constant regions (SEQ ID NOs.: 51 and 52), respectively. The heavy chain variable region and the light chain variable region had amino acid sequences set forth in SEQ ID NOs.: 13 and 27 for D2H3, and set forth in SEQ ID NOs.: 22 and 34 for D2A4. The activities of the resulting chimeric antibodies were confirmed in binding capture ELISA, competition ELISA and cell-based functional reporter assay following the protocols in the foregoing Examples. The data showed that the chimeric D2H3 and D2A4 antibodies had comparable activities to their respective mouse versions, as shown in Table 6 below.

TABLE 6
Binding and functional activities of Recombinant Chimeric Antibodies

| | | PD1/PDL1 ligand | Benchmark | Cell-based |
|---|---|---|---|---|
| | Capture binding | blocking ELISA | blocking ELISA | functional reporter |
| Clone ID# | ELISA (EC₅₀, nM) | (IC₅₀, nM) | (IC₅₀, nM) | assay (IC₅₀, nM) |
| mouse D2H3 | 0.17 | 0.89 | 0.21 | 8.48 |
| chimeric D2H3 | 0.17 | 0.74 | 0.19 | 5.36 |
| mouse D2A4 | 0.13 | 1.51 | 0.33 | 6.18 |
| chimeric D2A4 | 0.24 | 1.71 | 0.35 | 9.46 |

### Example 6 Humanization of Anti-PD-1 Mouse Monoclonal Antibodies D2H3 and D2A4

Mouse anti-PD1 antibodies D2H3 and D2A4 were selected for humanization and further investigations. Humanization of the murine antibodies was conducted using the well-established CDR-grafting method as described in detail below.

To select acceptor frameworks for humanization of mouse antibodies D2H3 and D2A4, the light and heavy chain variable region sequences of D2H3 and D2A4 were blasted against the human immunoglobulin gene database. The human germline IGVH and IGVK with the highest homology to D2H3 and D2A4 were selected as the acceptor frameworks for humanization. The mouse antibody heavy/light chain variable region CDRs were inserted the selected frameworks, and the residue(s) in the frameworks was/were backmutated to obtain more candidate heavy chain/light chain variable regions. The humanized D2H3 and D2A4 variable heavy and light chain variants were designed as shown in Table 7 and Table 8 below, respectively.

TABLE 7
Design of D2H3 variable heavy and light humanized variants

| | Backmutation(s) |
|---|---|
| VH and VL variant | (Chothia#) |
| VH for huD2H3-V1, huD2H3- | None |
| V8- huD2H3-V11 and | |
| huD2H3-V13 | |
| VH for huD2H3-V2 | S24A |
| VH for huD2H3-V3 | I48M |
| VH for huD2H3-V4 | A67V |
| VH for huD2H3-V5 | L691 |
| VH for huD2H3-V6 | T73E |
| VH for huD2H3-V7 | F91Y |
| VH for huD2H3-V12 and | S24A, I48M, A67V, |
| huD2H3-V14 | L691, T73E, F91Y |
| VL for huD2H3-V1 - huD2H3-V7 | None |
| and huD2H3-V12 | |
| VL for huD2H3-V8 | L4M |
| VL for huD2H3-V9 | S49Y |
| VL for huD2H3-V10 | E68G |
| VL for huD2H3-V11 | F87Y |
| VL for huD2H3-V13 and | L4M, S49Y, E68G, |
| huD2H3-V14 | F87Y |

TABLE 8
Design of D2A4 variable heavy and light humanized variants

| | Backmutation(s) |
|---|---|
| VH and VL variant | (Chothia#) |
| VH for huD2A4-V1 and huD2A4-V4 | None |
| VH for huD2A4-V2 | A49S |
| VH for huD2A4-V3 | A105K |
| VH for huD2A4-V5 and huD2A4-V6 | A49S, A105K |
| VL for huD2A4-V1 - huD2A4-V3 and | None |
| huD2A4-V5 | |
| VL for huD2A4-V4 and huD2A4-V6 | S43A |

The vectors containing nucleoride sequences encoding humanized D2H3/D2A4 heavy chain/light chain variable regions and human IgG1 heavy-chain and human kappa light-chain constant regions were transiently transfected into 50 ml of 293F suspension cell cultures in a ratio of 60% to 40% light to heavy chain construct, with 1.2 mg/ml PEI. Cell supernatants were harvested after six days in shaking flasks, spun down to pellet cells, and filtered through 0.22 μm filters before IgG separation. The antibodies were purified by protein A affinity chromatography. Briefly, Protein A sepharose column (from bestchrom (Shanghai) Biosciences, Cat #AA0273) was washed using PBS buffer in 5 to 10 column volumes. Cell supernatants were passed through the columns, and then the columns were washed using PBS buffer until the absorbance for protein reached the baseline. The columns were eluted with elution buffer (0.1 M Glycine-HCl, pH 2.7), and immediately collected into 1.5 ml tubes with neutralizing buffer (1 M Tris-HCl, pH 9.0). Fractions containing IgG were pooled and dialyzed in PBS overnight at 4° C.

A total of 14 humanized antibodies (from huD2H3-V1 to huD2H3-V14) were obtained for D2H3, and 6 (from huD2A4-V1 to huD2A4-V6) for D2A4. The heavy chain/light chain variable region amino acid sequences of these antibodies were summarized in Table 1 above, the human IgG1 heavy-chain and human kappa light-chain constant region sequences were set forth in SEQ ID NOs.: 51 and 52, respectively.

The binding affinity of the obtained humanized antibodies were assessed for their binding activities to human PD1 through a capture binding ELISA as describe in Example 3, and the binding EC₅₀ values were summarized in Tables 9.1-9.3 and 10.1-10.2. 14 humanized D2H3 antibodies and 6 humanized D2A4 antibodies had comparable affinities to the chimeric antibodies D2H3 (chD2H3) and D2A4 (chD2A4), respectively.

TABLE 9.1
Binding Activities of Humanized D2H3 Antibodies huD2H3-V1 to huD2H3-V6

| | | | | | | | | | | Mouse |
|---|---|---|---|---|---|---|---|---|---|---|
| | | | | | | | | | | IgG-NC/ |
| Capture | Mouse | | huD2H3- | huD2H3- | huD2H3- | huD2H3- | huD2H3- | huD2H3- | | Human |
| ELISA | D2H3 | chD2H3 | V1 | V2 | V3 | V4 | V5 | V6 | OPDIVO ® | IgG-NC |
| EC₅₀ | 0.18 | 1.04 | 0.28 | 0.21 | 0.19 | 0.28 | 0.32 | 0.17 | 0.27 | — |
| (ng/mL) | | | | | | | | | | |

TABLE 9.2
Binding Activities of Humanized D2H3 Antibodies huD2H3-V7 to huD2H3-V11

| | | | | | | | | | | Mouse |
|---|---|---|---|---|---|---|---|---|---|---|
| | | | | | | | | | | IgG-NC/ |
| Capture | Mouse | Mouse | | huD2H3- | huD2H3- | huD2H3- | huD2H3- | huD2H3- | | Human |
| ELISA | D2H3 | IgG-NC | chD2H3 | V7 | V8 | V9 | V10 | V11 | OPDIVO ® | IgG-NC |
| EC₅₀ | 0.229 | — | 1.511 | 0.157 | 0.171 | 0.358 | 0.505 | 0.170 | 0.365 | — |
| (ng/mL) | | | | | | | | | | |

TABLE 9.3
Binding Activities of Humanized D2H3 Antibodies huD2H3-V12 and huD2H3-V14

| Capture | Mouse | Mouse | | huPD1- | huD2H3- | | Human |
|---|---|---|---|---|---|---|---|
| ELISA | D2H3 | IgG-NC | chD2H3 | D2H3-V12 | V14 | OPDIVO ® | IgG-NC |
| EC₅₀ | 0.388 | — | 0.310 | 0.354 | 0.531 | 1.374 | — |
| (ng/mL) | | | | | | | |

TABLE 10.1
Binding Activities of Humanized D2A4 Antibodies huD2A4-V1 to huD2A4-V4

| | | | | | | | | Mouse |
|---|---|---|---|---|---|---|---|---|
| | | | | | | | | IgG-NC/ |
| Capture | Mouse | | huD2A4- | huD2A4- | huD2A4- | huD2A4- | | Human |
| ELISA | D2A4 | ChD2A4 | V1 | V2 | V3 | V4 | OPDIVO ® | IgG-NC |
| EC₅₀ | 0.281 | 0.681 | 0.385 | 0.379 | 0.550 | 0.349 | 0.351 | — |
| (ng/mL) | | | | | | | | |

TABLE 10.2
Binding Activities of Humanized
D2A4 Antibodies huD2A4-V5 and huD2A4-V6

| Capture | | huD2A4- | huD2A4- | | Human |
|---|---|---|---|---|---|
| ELISA | ChD2A4 | V5 | V6 | OPDIVO ® | IgG-NC |
| EC₅₀ | 0.773 | 0.773 | 0.999 | 0.268 | — |
| (ng/mL) | | | | | |

The results indicated that the humanized D2H3 and D2A4 antibodies bound to human PD-1 specifically, with several ones showing similar or lower EC₅₀ values compared to Nivolumab.

The humanized antibodies huD2H3-V14 and huD2A4-V6 were then tested for their affinities for human and cynomolgus PD1 by Biacore and by binding capture ELISA, and also tested for their functional activities by competition ELISA and by cell-based reporter assay, following the protocols in Examples 2 to 4. As showed in Table 11, both huD2H3-V14 and huD2A4-V6 showed comparable in vitro activities compared to their corresponding mouse antibodies, and thus increased affinity to PD-1 and improved functions compared to OPDIVO®.

TABLE 11
Binding and Functional activities of Humanized D2H3 and D2A4 mAbs
Summary of In Vitro binding and functional activities of humanized PD1 mAbs

| | | | | Functional assay | | |
|---|---|---|---|---|---|---|
| | Binding assay | | | Competition ELISA (IC₅₀, nM) | | Cell- |
| | Human PD1 | | Cynomolg | PD1/PDL1 | Benchmark | based |
| | Binding | | us PD1 | ligand blocking | blocking | functional |
| | ELISA | Biacore | Biacore | ELISA | ELISA | assay |
| mAbs | (EC₅₀, nM) | (KD, M) | (KD, M) | (IC₅₀, nM) | (IC₅₀, nM) | (EC₅₀, nM) |
| huD2A4-V6 | 0.27 | 3.06E−09 | 6.43E−09 | 1.69 | 0.49 | 7.60 |
| chD2A4 | 0.24 | Not tested | Not tested | 1.71 | 0.35 | 9.46 |
| mouse D2A4 | 0.13 | 2.55E−09 | 6.57E−09 | 1.51 | 0.33 | 6.18 |
| huD2H3-V14 | 0.17 | 3.08E−10 | 1.50E−09 | 0.68 | 0.18 | 7.11 |
| chD2H3 | 0.17 | 4.41E−10 | 1.78E−09 | 0.74 | 0.19 | 5.36 |
| mouse D2H3 | 0.17 | 3.73E−10 | 1.46E−09 | 0.89 | 0.21 | 8.48 |
| OPDIVO ® | 0.45 | 8.17E−09 | 1.40E−08 | 0.42 | 0.39 | 10.32 |

### Example 7 In Vivo Anti-Tumor Efficacy of Humanized D2H3 and D2A4 Antibodies

The effect of humanized anti-PD-1 antibodies on tumor growth was evaluated on MC38 xenograft model. Briefly, female B-hPD-1 plus mice of 5-8 weeks old were subcutaneously injected with 5×10⁵ MC38 cells at the right hind flank. When tumor volumes reached about 100-150 mm³, mice were randomly divided into 10 groups, 8 mice/group. On the same day (referred to as Day 0), the animals began to take drug administration. Specifically, the mice were intraperitoneally injected with PBS, huD2A4-V6, huD2H3-V14 and OPDIVO®, respectively, at a dose of 1 mg/kg, 3 mg/kg or 10 mg/kg, twice a week for three weeks. The detailed dosing scheme was shown in Table 12 below. The humanized antibodies huD2A4-V6 and huD2H3-V14 contained heavy chain and light chain constant regions having amino acid sequences set forth in SEQ ID NOs: 51 and 52, respectively. Mice body weights and tumor volumes were measured and recorded twice a week. The tumor volume (V) was calculated as (length×width²)/2.

TABLE 12
Dosing Scheme

| Group No. | Drug | Treatment Group | |
|---|---|---|---|
| 1 | PBS | n/a | |
| 2 | huD2A4-V6 | 1 | mg/kg |
| 3 | huD2A4-V6 | 3 | mg/kg |
| 4 | huD2A4-V6 | 10 | mg/kg |
| 5 | huD2H3-V14 | 1 | mg/kg |
| 6 | huD2H3-V14 | 3 | mg/kg |
| 7 | huD2H3-V14 | 10 | mg/kg |
| 8 | OPDIVO ® | 1 | mg/kg |
| 9 | OPDIVO ® | 3 | mg/kg |
| 10 | OPDIVO ® | 10 | mg/kg |

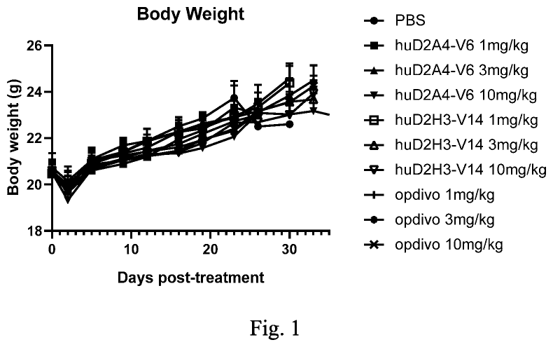

Mice body weights were shown in FIG. 1. No difference was found among groups, indicating that all treatments were well tolerated by the tumor-bearing animals.

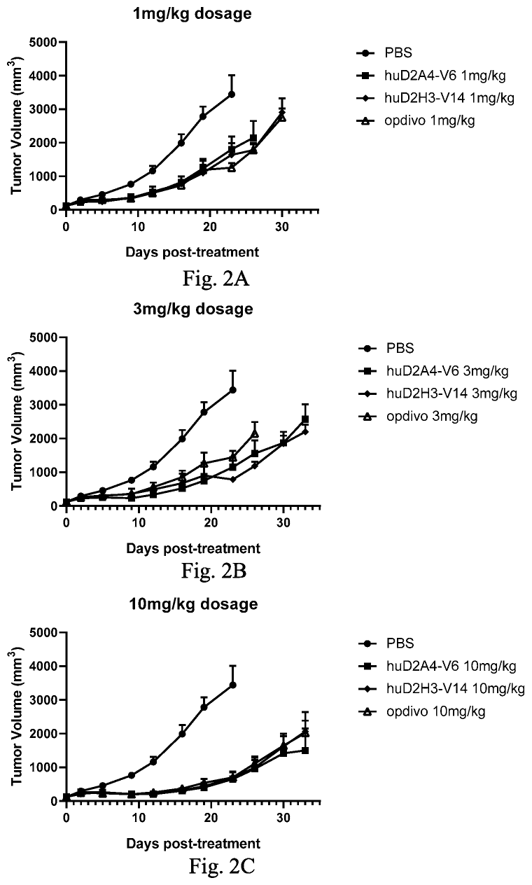

FIG. 2A-2C showed mice tumor volume change in groups administed with different agents at different doses. Mice administed with huD2A4-V6, huD2H3-V14 and OPDIVO® at the 1 mg/kg dose level had significantly smaller tumor volumes than those in the vehicle control group (FIG. 2A). At the dose of 3 mg/kg, three agents all showed evident anti-tumor effects, and the tumor volumes in huD2A4-V6 and huD2H3-V14 groups were slightly smaller than the OPDIVO® group (FIG. 2B). Further, as shown in FIG. 2C, huD2A4-V6, huD2H3-V14 and OPDIVO® at the daily dose of 10 mg/kg provided similar anti-tumor effect at the first four weeks, but mice in huD2A4-V6group had slightly smaller tumor volumes than those in the other two treatment groups on Day 30-33. In huD2A4-V6 10 mg/kg group, the tumor totally vanished in one mouse, and the tumor stopped to grow in another mouse, which were not observed in animals from other groups.

While the invention has been described above in connection with one or more embodiments, it should be understood that the invention is not limited to those embodiments, and the description is intended to cover all alternatives, modifications, and equivalents, as may be included within the spirit and scope of the appended claims. All referenced cited herein are further incorporated by reference in their entirety.

Sequences in the present application are summarized below.

Description/
Sequence/SEQ ID NO.

VH-CDR1 for mouse, chimeric and humanized D2H3
GYTFTNYW (SEQ ID NO: 1) defined by IMGT numbering scheme
GYTFTNY (SEQ ID NO: 37) defined by Chothia numbering scheme
NYWMH (SEQ ID NO: 38) defined by Kabat numbering scheme
VH-CDR2 for mouse, chimeric and humanized D2H3
IFPRNSET (SEQ ID NO: 2) defined by IMGT numbering scheme
FPRNSE (SEQ ID NO: 39) defined by Chothia numbering scheme
A1FPRNSETNYNQKFKA (SEQ ID NO: 40) defined by Kabat numbering scheme
VH-CDR3 for mouse, chimeric and humanized D2H3
TRNRYGLDY (SEQ ID NO: 3) defined by IMGT numbering scheme
NRYGLDY (SEQ ID NO: 41) defined by Chothia or Kabat numbering scheme
VH-CDR1 for mouse, chimeric and humanized D2A4
GFTFSSYT (SEQ ID NO: 4) defined by IMGT numbering scheme
GFTFSSY (SEQ ID NO: 44) defined by Chothia numbering scheme
SYTMS (SEQ ID NO: 45) defined by Kabat numbering scheme
VH-CDR2 for mouse, chimeric and humanized D2A4
ISGGGSNT (SEQ ID NO: 5) defined by IMGT numbering scheme
SGGGSN (SEQ ID NO: 46) defined by Chothia numbering scheme
TISGGGSNTYYPDSVKG (SEQ ID NO: 47) defined by Kabat numbering scheme
VH-CDR3 for mouse, chimeric and humanized D2A4
ARQAFYSNYWYFDV (SEQ ID NO: 6) defined by IMGT numbering scheme
QAFYSNYWYFDV (SEQ ID NO: 48) defined by Chothia or Kabat numbering scheme
VL-CDR1 for mouse, chimeric and humanized D2H3
RASESVSLHGTRLMH (SEQ ID NO: 7) defined by Chothia or Kabat numbering scheme
ESVSLHGTRL (SEQ ID NO: 42) defined by IMGT numbering scheme
VL-CDR2 for mouse, chimeric and humanized D2H3
LGSNLES (SEQ ID NO: 8) defined by Chothia or Kabat numbering scheme
LGS (SEQ ID NO: 43) defined by IMGT numbering scheme
VL-CDR3 for mouse, chimeric and humanized D2H3
QQSIEDPWT (SEQ ID NO: 9) defined by IMGT numbering scheme, Chothia
numbering scheme or Kabat numbering scheme
VL-CDR1 for mouse, chimeric and humanized D2A4
LASQTIGTWLA (SEQ ID NO: 10) defined by Chothia or Kabat numbering scheme
QTIGTW (SEQ ID NO: 49) defined by IMGT numbering scheme
VL-CDR2 for mouse, chimeric and humanized D2A4
AATSLAD (SEQ ID NO: 11) defined by Chothia or Kabat numbering scheme
AAT (SEQ ID NO: 50) defined by IMGT numbering scheme
VL-CDR3 for mouse, chimeric and humanized D2A4
QQVSSIPWT (SEQ ID NO: 12) defined by IMGT numbering scheme, Chothia
numbering scheme or Kabatnumbering scheme
VH for mouse D2H3
EVQLQQSGTVLARPGASVKMSCKSSGYTFTNYWMHWVKQRPGQGLEWIGAIFPRNSETNYNQKFKAK
AKLTAVTSASTAYMEVSSLTSEDSAVYFCTRNRYGLDYWGQGTSVTVSS (SEQ ID NO: 13)
GAGGTTCAGCTCCAGCAGTCTGGGACTGTGCTGGCAAGGCCTGGGGCCTCAGTGAAGATGTCCTGC
AAGTCTTCTGGCTACACCTTTACCAACTACTGGATGCACTGGGTAAAACAGAGGCCTGGACAGGGT
CTGGAATGGATTGGCGCTATTTTTCCTAGAAATAGTGAGACTAACTACAACCAGAAATTTAAGGCC
AAGGCCAAACTGACTGCAGTCACATCTGCCAGCACTGCCTACATGGAGGTCAGCAGCCTGACAAGT
GAGGACTCTGCGGTCTATTTCTGTACGAGGAATAGGTATGGTCTGGACTACTGGGGTCAAGGAACC
TCAGTCACCGTCTCCTCA (SEQ IN NO: 55)
VH for huD2H3-V1, huD2H3-V8- huD2H3-V11 and huD2H3-V13
EVQLVQSGAEVKKPGSSVKVSCKSSGYTFTNYWMHWVRQAPGQGLEWIGAIFPRNSETNYNQKFKAR
ATLTADTSTSTAYMELSSLRSEDTAVYFCTRNRYGLDYWGQGTLVTVSS (SEQ ID NO: 14)
VH for huD2H3-V2
EVQLVQSGAEVKKPGSSVKVSCKASGYTFTNYWMHWVRQAPGQGLEWIGAIFPRNSETNYNQKFKAR
ATLTADTSTSTAYMELSSLRSEDTAVYFCTRNRYGLDYWGQGTLVTVSS (SEQ ID NO: 15)
VH for huD2H3-V3
EVQLVQSGAEVKKPGSSVKVSCKSSGYTFTNYWMHWVRQAPGQGLEWMGAIFPRNSETNYNQKFKA
RATLTADTSTSTAYMELSSLRSEDTAVYFCTRNRYGLDYWGQGTLVTVSS (SEQ ID NO: 16)
VH for huD2H3-V4
EVQLVQSGAEVKKPGSSVKVSCKSSGYTFTNYWMHWVRQAPGQGLEWIGAIFPRNSETNYNQKFKAR
VTLTADTSTSTAYMELSSLRSEDTAVYFCTRNRYGLDYWGQGTLVTVSS (SEQ ID NO: 17)
VH for huD2H3-V5
EVQLVQSGAEVKKPGSSVKVSCKSSGYTFTNYWMHWVRQAPGQGLEWIGAIFPRNSETNYNQKFKAR
ATITADTSTSTAYMELSSLRSEDTAVYFCTRNRYGLDYWGQGTLVTVSS (SEQ ID NO: 18)
VH for huD2H3-V6
EVQLVQSGAEVKKPGSSVKVSCKSSGYTFTNYWMHWVRQAPGQGLEWIGAIFPRNSETNYNQKFKAR
ATLTADESTSTAYMELSSLRSEDTAVYFCTRNRYGLDYWGQGTLVTVSS (SEQ ID NO: 19)
VH for huD2H3-V7
EVQLVQSGAEVKKPGSSVKVSCKSSGYTFTNYWMHWVRQAPGQGLEWIGAIFPRNSETNYNQKFKAR
ATLTADTSTSTAYMELSSLRSEDTAVYYCTRNRYGLDYWGQGTLVTVSS (SEQ ID NO: 20)
VH for huD2H3-V12 and huD2H3-V14
EVQLVQSGAEVKKPGSSVKVSCKASGYTFTNYWMHWVRQAPGQGLEWMGAIFPRNSETNYNQKFKA
RVTITADESTSTAYMELSSLRSEDTAVYYCTRNRYGLDYWGQGTLVTVSS (SEQ ID NO: 21)
GAGGTGCAGCTGGTGCAGAGCGGCGCCGAGGTGAAGAAGCCCGGCTCCAGCGTGAAGGTGAGCTG
CAAGGCCAGCGGGTACACCTTCACAAACTACTGGATGCACTGGGTGAGGCAGGCCCCAGGGCAGG
GCCTGGAGTGGATGGGCGCCATCTTCCCCAGGAACAGCGAGACAAACTACAACCAGAAGTTCAAG
GCCAGGGTGACAATCACAGCCGATGAGAGCACCAGCACAGCCTACATGGAGCTGAGCAGCCTGCG
GAGCGAGGACACCGCCGTGTACTACTGCACCCGGAACCGGTACGGGCTGGATTACTGGGGGCAGG
GGACCCTGGTGACAGTGAGCAGC (SEQ ID NO: 56)
VH for mouse D2A4
EVKLVESGGGLVKPGGSLKLSCAASGFTFSSYTMSWVRQTPAKRLEWVATISGGGSNTYYPDSVKGRF
TISRDNARNTLYLQMSSLRSEDTAMYYCARQAFYSNYWYFDVWGAGTTVTVSS (SEQ ID NO: 22)
GAAGTGAAGCTGGTGGAGTCTGGGGGAGGCTTAGTGAAGCCTGGAGGGTCCCTGAAACTCTCCTGT
GCAGCCTCTGGATTCACTTTCAGTAGCTATACCATGTCTTGGGTTCGCCAGACTCCGGCGAAGAGG
CTGGAGTGGGTCGCAACCATTAGTGGAGGTGGTAGTAACACCTACTATCCTGACAGTGTGAAGGGC
CGATTCACCATCTCCAGAGACAATGCCAGGAACACCCTGTACCTGCAAATGAGCAGTCTGAGGTCT
GAGGACACGGCCATGTATTACTGTGCAAGACAAGCCTTCTATAGTAACTACTGGTACTTCGACGTC
TGGGGCGCAGGGACCACGGTCACCGTCTCCTCA (SEQ ID NO: 57)
VH for huD2A4-V1 and huD2A4-V4
EVQLVESGGGLVQPGGSLRLSCAASGFTFSSYTMSWVRQAPGKGLEWVATISGGGSNTYYPDSVKGRF
TISRDNSKNTLYLQMNSLRAEDTAVYYCARQAFYSNYWYFDVWGAGTTVTVSS (SEQ ID NO: 23)
VH for huD2A4-V2
EVQLVESGGGLVQPGGSLRLSCAASGFTFSSYTMSWVRQAPGKGLEWVSTISGGGSNTYYPDSVKGRF
TISRDNSKNTLYLQMNSLRAEDTAVYYCARQAFYSNYWYFDVWGAGTTVTVSS (SEQ ID NO: 24)
VH for huD2A4-V3
EVQLVESGGGLVQPGGSLRLSCAASGFTFSSYTMSWVRQAPGKGLEWVATISGGGSNTYYPDSVKGRF
TISRDNSKNTLYLQMNSLRAEDTAVYYCARQAFYSNYWYFDVWGKGTTVTVSS (SEQ ID NO: 25)
VH for huD2A4-V5 and huD2A4-V6
EVQLVESGGGLVQPGGSLRLSCAASGFTFSSYTMSWVRQAPGKGLEWVSTISGGGSNTYYPDSVKGRF
TISRDNSKNTLYLQMNSLRAEDTAVYYCARQAFYSNYWYFDVWGKGTTVTVSS (SEQ ID NO: 26)
GAGGTGCAGCTGGTGGAGAGCGGGGGGGGACTGGTGCAGCCAGGAGGAAGCCTGAGACTGAGCT
GTGCCGCAAGCGGGTTCACATTTAGTAGTTACACAATGAGCTGGGTGAGACAGGCCCCCGGAAAA
GGACTGGAGTGGGTGTCTACTATTTCAGGAGGGGGAAGCAACACCTATTATCCCGATAGTGTGAAG
GGCAGATTCACAATCAGTAGAGACAACAGCAAGAACACCCTGTACCTGCAGATGAACAGCCTGAG
AGCCGAGGACACCGCCGTGTACTACTGCGCCAGACAGGCCTTTTACAGTAACTATTGGTACTTCGA
CGTGTGGGGAAAGGGAACCACAGTGACTGTGAGCAGC (SEQ ID NO: 58)
VL for mouse D2H3
DIVLTQSPGFLAVSLGQRATISCRASESVSLHGTRLMHWYHQKPGQPPKLLISLGSNLESGVPARFSGSG
SETDFTLNIFIPVEEEDAATYFCQQSIEDPWTFGGGTKLEIK (SEQ ID NO: 27)
GACATCGTGCTGACCCAATCTCCAGGTTTTTTGGCTGTGTCTCTAGGGCAGAGGGCCACCATCTCCT
GCAGAGCCAGTGAAAGTGTCAGTCTTCATGGTACTCGTTTAATGCACTGGTACCATCAGAAACCAG
GACAGCCACCCAAACTCCTCATCTCTCTTGGATCCAACCTAGAGTCTGGAGTCCCTGCCAGGTTCA
GTGGCAGTGGGTCTGAGACAGACTTCACCCTCAACATCCATCCTGTGGAGGAGGAGGATGCTGCAA
CCTATTTCTGTCAGCAAAGTATTGAGGATCCGTGGACGTTCGGTGGAGGCACCAAGCTGGAAATCA
AA (SEQ ID NO: 59)
VL for huD2H3-V1 - huD2H3-V7 and huD2H3-V12
DIVLTQSPDSLAVSLGERATINCRASESVSLHGTRLMHWYQQKPGQPPKLLISLGSNLESGVPDRFSGSG
SETDFTLTISSLQAEDVAVYFCQQSIEDPWTFGQGTKLEIK (SEQ ID NO: 28)
VL for huD2H3-V8
DIVMTQSPDSLAVSLGERATINCRASESVSLHGTRLMHWYQQKPGQPPKLLISLGSNLESGVPDRFSGSG
SETDFTLTISSLQAEDVAVYFCQQSIEDPWTFGQGTKLE1K (SEQ ID NO: 29)
VL for huD2H3-V9
DIVLTQSPD SLAVSLGERATINCRASESVSLHGTRLMHWYQQKPGQPPKLLIYLGSNLESGVPDRFSGSG
SETDFTLTISSLQAEDVAVYFCQQSIEDPWTFGQGTKLEIK (SEQ ID NO: 30)
VL for huD2H3-V10
DIVLTQSPDSLAVSLGERATINCRASESVSLHGTRLMHWYQQKPGQPPKLLISLGSNLESGVPDRFSGSG
SGTDFTLTISSLQAEDVAVYFCQQSIEDPWTFGQGTKLEIK (SEQ ID NO: 31)
VL for huD2H3-V11
DIVLTQSPDSLAVSLGERATINCRASESVSLHGTRLMHWYQQKPGQPPKLLISLGSNLESGVPDRFSGSG
SETDFTLTISSLQAEDVAVYYCQQSIEDPWTFGQGTKLEIK (SEQ ID NO: 32)
VL for huD2H3-V13 and huD2H3-V14
DIVMTQSPDSLAVSLGERATINCRASESVSLHGTRLMHWYQQKPGQPPKLLIYLGSNLESGVPDRFSGS
GSGTDFTLTISSLQAEDVAVYYCQQSIEDPWTFGQGTKLEIK (SEQ ID NO: 33)
GATATCGTGATGACACAGAGCCCCGATAGCCTGGCCGTGAGCCTGGGGGAGCGGGCCACCATCAA
CTGCCGGGCCAGCGAGAGCGTGAGCCTGCACGGGACAAGGCTGATGCACTGGTACCAGCAGAAGC
CCGGCCAGCCCCCCAAGCTGCTGATCTACCTGGGGAGCAACCTGGAGAGCGGGGTGCCCGATAGG
TTCAGCGGCAGCGGGAGCGGCACAGATTTCACACTGACCATCAGCAGCCTGCAGGCCGAGGACGT
GGCCGTGTACTACTGCCAGCAGAGCATCGAGGACCCCTGGACCTTCGGCCAGGGCACAAAGCTGG
AGATCAAG (SEQ ID NO: 60)
VL for mouse D2A4
DIQMTQSPASQSASLGESVTITCLASQTIGTWLAWYQQTPGKSPQLLIYAATSLADGVPSRFSGSGSGTK
FSFKISSLQAEDFASYYCQQVSS1PWTFGGGTKLEIR (SEQ ID NO: 34)
GACATTCAGATGACCCAGTCTCCTGCCTCCCAGTCTGCATCTCTGGGAGAAAGTGTCACCATCACA
TGCCTGGCAAGTCAGACCATTGGTACATGGTTAGCATGGTATCAGCAGACACCAGGGAAATCTCCT
CAGCTCCTGATTTATGCTGCAACCAGCTTGGCAGATGGGGTCCCATCAAGGTTCAGTGGTAGTGGA
TCTGGCACAAAGTTTTCTTTCAAGATCAGCAGCCTACAGGCTGAAGATTTTGCAAGTTATTACTGTC
AACAAGTTTCCAGTATTCCGTGGACGTTCGGTGGAGGCACCAAGCTGGAAATCAGA (SEQ ID NO:
61)
VL for huD2A4-V1 - huD2A4-V3 and huD2A4-V5
DIQMTQSPSSVSASVGDRVTITCLASQTIGTWLAWYQQKPGKSPKLLIYAATSLADGVPSRFSGSGSGTD
FTLTISSLQPEDFATYYCQQVSSIPWTFGGGTKVEIK (SEQ ID NO: 35)
VL for huD2A4-V4 and huD2A4-V6
DIQMTQSPSSVSASVGDRVTITCLASQTIGTWLAWYQQKPGKAPKLLIYAATSLADGVPSRFSGSGSGT
DFTLTISSLQPEDFATYYCQQVSSIPWTFGGGTKVEIK (SEQ ID NO: 36)
GACATTCAGATGACCCAGAGCCCCAGCAGCGTGAGCGCCAGCGTGGGAGACAGAGTGACCATAAC
CTGCCTGGCCAGCCAAACCATAGGCACCTGGCTGGCCTGGTACCAGCAGAAACCCGGCAAAGCCC
CCAAACTGCTCATCTACGCCGCCACCAGCCTGGCTGACGGAGTGCCAAGCAGATTCTCCGGTAGCG
GCAGCGGCACCGACTTCACCCTGACTATCAGCAGCCTCCAACCCGAAGACTTCGCCACCTACTACT
GCCAACAGGTCTCCTCCATTCCCTGGACCTTCGGAGGCGGCACCAAGGTGGAGATCAAA (SEQ ID
NO: 62)
human IgG1 heavy chain constant region
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSLSPGK (SEQ ID NO: 51)
GCTAGCACCAAGGGCCCATCGGTCTTCCCCCTGGCACCCTCCTCCAAGAGCACCTCTGGGGGCACA
GCGGCCCTGGGCTGCCTGGTCAAGGACTACTTCCCCGAACCGGTGACGGTGTCGTGGAACTCAGGC
GCCCTGACCAGCGGCGTGCACACCTTCCCGGCTGTCCTACAGTCCTCAGGACTCTACTCCCTCAGCA
GCGTGGTGACCGTGCCCTCCAGCAGCTTGGGCACCCAGACCTACATCTGCAACGTGAATCACAAGC
CCAGCAACACCAAGGTGGACAAGAAAGTTGAGCCCAAATCTTGTGACAAAACTCACACATGCCCAC
CGTGCCCAGCACCTGAACTCCTGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAAT
GGCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGAGGAGATGAC
CAAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCT
CCTTCTTCCTCTACAGCAAGCTCACCGTGGACAAGAGCAGGTGGCAGCAGGGGAACGTCTTCTCAT
GCTCCGTGATGCATGAGGCTCTGCACAACCACTACACGCAGAAGAGCCTCTCCCTGTCTCCGGGTAA
A (SEQ lD NO: 63)
human kappa light chain constant region
RTVAAPSVFIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC (SEQ ID NO: 52)
CGTACGGTGGCGGCGCCATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCAAGGACAGCACCTACA
GCCTCAGCAGCACCCTGACGCTGAGCAAAGCAGACTACGAGAAACACAAAGTCTACGCCTGCGAA
GTCACCCATCAGGGCCTGAGCTCGCCCGTCACAAAGAGCTTCAACAGGGGAGAGTGT (SEQ ID NO:
64)
human PD-1-Fc
LDSPDRPWNPPTFSPALLVVTEGDNATFTCSFSNTSESFVLNWYRMSPSNQTDKLAAFPEDRSQPGQDC
RFRVTQLPNGRDFHMSVVRARRNDSGTYLCGAISLAPKAQIKESLRAELRVTERRAEVPTAHPSPSPRP
AGQFQEPKSCDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSL (SEQ ID NO: 53)
human PD-L1-Fc
FTVTVPKDLYVVEYGSNMTIECKFPVEKQLDLAALIVYWEMEDKNIIQFVHGEEDLKVQHSSYRQRAR
LLKDQLSLGNAALQITDVKLQDAGVYRCMISYGGADYKRITVKVNAPYNKINQRILVVDPVTSEHELT
CQAEGYPKAEVIWTSSDHQVLSGKTTTTNSKREEKLFNVTSTLRINTTTNEIFYCTFRRLDPEENHTAEL
VIPELPLAHPPNERTEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK (SEQ ID NO: 54)
mouse IgG1 heavy chain constant region
AKTTPPSVYPLAPGSAAQTNSMVTLGCLVKGYFPEPVTVTWNSGSLSSGVHTFPAVLQSDLYTLSSSVT
VPSSTWPSETVTCNVAHPASSTKVDKKIVPRDCGCKPCICTVPEVSSVFIFPPKPKDVLTITLTPKVTCVV
VDISKDDPEVQFSWFVDDVEVHTAQTQPREEQFNSTFRSVSELPIMHQDWLNGKEFKCRVNSAAFPAPI
EKTISKTKGRPKAPQVYTIPPPKEQMAKDKVSLTCMITDFFPEDITVEWQWNGQPAENYKNTQPIMDTD
GSYFVYSKLNVQKSNWEAGNTFTCSVLHEGLHNHHTEKSLSHSPGK (SEQ ID NO: 65)
GCCAAAACGACACCCCCATCTGTCTATCCACTGGCCCCTGGATCTGCTGCCCAAACTAACTCCATG
GTGACCCTGGGATGCCTGGTCAAGGGCTATTTCCCTGAGCCAGTGACAGTGACCTGGAACTCTGGA
TCCCTGTCCAGCGGTGTGCACACCTTCCCAGCTGTCCTGCAGTCTGACCTCTACACTCTGAGCAGCT
CAGTGACTGTCCCCTCCAGCACCTGGCCCAGCGAGACCGTCACCTGCAACGTTGCCCACCCGGCCA
GCAGCACCAAGGTGGACAAGAAAATTGTGCCCAGGGATTGTGGTTGTAAGCCTTGCATATGTACAG
TCCCAGAAGTATCATCTGTCTTCATCTTCCCCCCAAAGCCCAAGGATGTGCTCACCATTACTCTGAC
TCCTAAGGTCACGTGTGTTGTGGTAGACATCAGCAAGGATGATCCCGAGGTCCAGTTCAGCTGGTT
TGTAGATGATGTGGAGGTGCACACAGCTCAGACGCAACCCCGGGAGGAGCAGTTCAACAGCACTT
TCCGCTCAGTCAGTGAACTTCCCATCATGCACCAGGACTGGCTCAATGGCAAGGAGTTCAAATGCA
GGGTCAACAGTGCAGCTTTCCCTGCCCCCATCGAGAAAACCATCTCCAAAACCAAAGGCAGACCG
AAGGCTCCACAGGTGTACACCATTCCACCTCCCAAGGAGCAGATGGCCAAGGATAAAGTCAGTCT
GACCTGCATGATAACAGACTTCTTCCCTGAAGACATTACTGTGGAGTGGCAGTGGAATGGGCAGCC
AGCGGAGAACTACAAGAACACTCAGCCCATCATGGACACAGATGGCTCTTACTTCGTCTACAGCAA
GCTCAATGTGCAGAAGAGCAACTGGGAGGCAGGAAATACTTTCACCTGCTCTGTGTTACATGAGGG
CCTGCACAACCACCATACTGAGAAGAGCCTCTCCCACTCTCCTGGTAAA (SEQ ID NO: 67)
Mouse kappa light chain constant region
RADAAPTVSIFPPSSEQLTSGGASVVCFLNNFYPKDINVKWKIDGSERQNGVLNSWTDQDSKDSTYSMS
STLTLTKDEYERHNSYTCEATHKTSTSPIVKSFNRGEC (SEQ ID NO: 66)
CGGGCTGATGCTGCACCAACTGTATCCATCTTCCCACCATCCAGTGAGCAGTTAACATCTGGAGGT
GCCTCAGTCGTGTGCTTCTTGAACAACTTCTACCCCAAAGACATCAATGTCAAGTGGAAGATTGAT
GGCAGTGAACGACAAAATGGCGTCCTGAACAGTTGGACTGATCAGGACAGCAAAGACAGCACCTA
CAGCATGAGCAGCACCCTCACGTTGACTAAGGACGAGTATGAACGACATAACAGCTATACCTGTG
AGGCCACTCACAAGACATCAACTTCACCCATTGTCAAGAGCTTCAACAGGGGAGAGTGT (SEQ ID
NO: 68)
SEQ ID NOs:1-54, 65 and 66: amino acid sequence; SEQ ID NOs: 55-64,
67 and 68: nucleotide sequence

1. An isolated monoclonal antibody, or an antigen-binding fragment thereof, comprising a heavy chain variable region comprising a CDR1 region, a CDR2 region and a CDR3 region, wherein the CDR1 region, the CDR2 region and the CDR3 region comprise amino acid sequences having at least 80%, 85%, 90%, 91%, 92%, 93%, 94%, 95%, 96%, 97%, 98%, 99% or 100% identity to
(1.1) SEQ ID NOs: 1, 2 and 3, respectively; or
(1.2) SEQ ID NOs: 4, 5 and 6, respectively, when defined by IMGT numbering scheme;
(2.1) SEQ ID NOs: 37, 39 and 41, respectively, or
(2.2) SEQ ID NOs: 44, 46 and 48, respectively, when defined by Chothia numbering scheme; or
(3.1) SEQ ID NOs: 38, 40 and 41, respectively, or
(3.2) SEQ ID NOs: 45, 47 and 48, respectively, when defined by Kabat numbering scheme;
wherein the isolated monoclonal antibody or the antigen-binding fragment thereof binds PD-1.

2. The isolated monoclonal antibody, or the antigen-binding fragment thereof, of claim 1, comprising a heavy chain variable region comprising an amino acid sequence having at least 80%, 85%, 90%, 91%, 92%, 93%, 94%, 95%, 96%, 97%, 98%, 99% or 100% identity to SEQ ID NOs: 13, 14, 15, 16, 17, 18, 19, 20, 21, 22, 23, 24, 25, or 26.

3. The isolated monoclonal antibody, or the antigen-binding fragment thereof, of claim 1, further comprising a light chain variable region comprising a CDR1 region, a CDR2 region and a CDR3 region, wherein the CDR1 region, the CDR2 region and the CDR3 region comprise amino acid sequences having at least 80%, 85%, 90%, 91%, 92%, 93%, 94%, 95%, 96%, 97%, 98%, 99% or 100% identity to
(1.1) SEQ ID NOs: 7, 8 and 9, respectively; or
(1.2) SEQ ID NOs: 10, 11 and 12, respectively, when defined by Kabat numbering scheme or Chothia numbering scheme, or
(2.1) SEQ ID NOs: 42, 43 and 9, respectively, or
(2.2) SEQ ID NOs: 49, 50 and 12, respectively, when defined by IMGT numbering scheme.

4. The isolated monoclonal antibody, or the antigen-binding fragment thereof, of claim 1, further comprising a light chain variable region comprising an amino acid sequence having at least 80%, 85%, 90%, 91%, 92%, 93%, 94%, 95%, 96%, 97%, 98%, 99% or 100% identity to SEQ ID NO: 27, 28, 29, 30, 31, 32, 33, 34, 35, or 36.

5. The isolated monoclonal antibody, or the antigen-binding fragment thereof, of claim 1, comprising a heavy chain variable region and a light chain variable region, wherein the heavy chain and the light chain variable regions comprise amino acid sequences having at least 80%, 85%, 90%, 91%, 92%, 93%, 94%, 95%, 96%, 97%, 98%, 99% or 100% identity to (1) SEQ ID NOs: 13 and 27, respectively; (2) SEQ ID NOs: 14 and 28, respectively; (3) SEQ ID NOs: 15 and 28, respectively; (4) SEQ ID NOs: 16 and 28, respectively; (5) SEQ ID NOs: 17 and 28, respectively; (6) SEQ ID NOs: 18 and 28, respectively; (7) SEQ ID NOs: 19 and 28, respectively; (8) SEQ ID NOs: 20 and 28, respectively; (9) SEQ ID NOs: 14 and 29, respectively; (10) SEQ ID NOs: 14 and 30 respectively; (11) SEQ ID NOs: 14 and 31, respectively; (12) SEQ ID NOs: 14 and 32, respectively; (13) SEQ ID NOs: 21 and 28, respectively; (14) SEQ ID NOs: 14 and 33, respectively; (15) SEQ ID NOs: 21 and 33, respectively; (16) SEQ ID NOs: 22 and 34, respectively; (17) SEQ ID NOs: 23 and 35, respectively; (18) SEQ ID NOs: 24 and 35, respectively; (19) SEQ ID NOs: 25 and 35, respectively; (20) SEQ ID NOs: 23 and 36, respectively; (21) SEQ ID NOs: 26 and 35, respectively; or (22) SEQ ID NOs: 26 and 36, respectively,

6. The isolated monoclonal antibody, or the antigen-binding fragment thereof, of claim 1, further comprising a heavy chain constant region and a light chain constant region.

7. The isolated monoclonal antibody, or the antigen-binding fragment thereof, of claim 6, comprising a heavy chain constant region having an amino acid sequence having at least 80%, 85%, 90%, 91%, 92%, 93%, 94%, 95%, 96%, 97%, 98%, 99% or 100% identity to SEQ ID NO: 51, and/or a light chain constant region having an amino acid sequence having at least 80%, 85%, 90%, 91%, 92%, 93%, 94%, 95%, 96%, 97%, 98%, 99% or 100% identity to SEQ ID NO: 52.

8. The isolated monoclonal antibody, or the antigen-binding fragment thereof, of claim 1, which (a) binds to human PD-1; (b) binds to monkey PD-1; (c) inhibits binding of PD-L1 to PD-1; (d) increases T cell proliferation; (e) stimulates an immune response; and/or (f) stimulates an antigen-specific T cell response.

9. The isolated monoclonal antibody, or the antigen-binding fragment thereof, of claim 1, which is a mouse, human, chimeric or humanized antibody.

10-13. (canceled)

14. A bispecific molecule, an immunoconjugate, a chimeric antigen receptor, an engineered T cell receptor, or an oncolytic virus, comprising the isolated monoclonal antibody, or the antigen-binding fragment thereof, of claim 1.

15. A pharmaceutical composition comprising the isolated monoclonal antibody, or the antigen-binding fragment thereof, of claim 1, and a pharmaceutically acceptable carrier.

16. The pharmaceutical composition of claim 15, further comprising an anti-tumor agent.

17. A method for the prevention and/or treatment of a cancer disease in a subject, comprising administering to the subject a therapeutically effective amount of the isolated monoclonal antibody, or the antigen-binding fragment thereof, of claim 1.

18. The method of claim 17, wherein the cancer disease is a solid or non-solid tumor.

19. The method of claim 17, wherein the cancer disease is lymphoma, leukemia, multiple myeloma, melanoma, colon adenocarcinoma, pancreas cancer, colon cancer, gastric intestine cancer, prostate cancer, bladder cancer, kidney cancer, ovary cancer, cervix cancer, breast cancer, lung cancer, renal-cell cancer, nasopharynx cancer, or a combination thereof.

20. The isolated monoclonal antibody, or the antigen-binding fragment thereof, of claim 1, wherein the antigen-binding fragment thereof is a Fab fragment, a F(ab′)2 fragment, a Fd fragment, a Fv fragment, a single chain Fv (scFv), or a nanobody.

21. The isolated monoclonal antibody, or the antigen-binding fragment thereof, of claim 8, which binds to PD-1 with a K_{D} of 1.0×10⁻⁸M or less and inhibiting the binding of PD-L1 to PD-1.

22. The isolated monoclonal antibody, or the antigen-binding fragment thereof, of claim 21, which binds to human PD-1 with a K_{D} of 0.3-4.0×10⁻⁹M or less and inhibiting the binding of PD-L1 to PD-1.

23. The isolated monoclonal antibody, or the antigen-binding fragment thereof, of claim 5, which is obtained from a mammalian cell.

24. The isolated monoclonal antibody, or the antigen-binding fragment thereof, of claim 23, wherein the mammalian cell is a CHO cell, a NSO myeloma cell, a COS cell or a SP2 cell.


SEQUENCE LISTING (68 sequences)

SEQ ID NO: 1 - protein, 8 aa, Artificial Sequence
  VH-CDR1 for mouse, chimeric and humanized D2H3, defined by IMGT numbering scheme
GYTFTNYW

SEQ ID NO: 2 - protein, 8 aa, Artificial Sequence
  VH-CDR2 for mouse, chimeric and humanized D2H3, defined by IMGT numbering scheme
IFPRNSET

SEQ ID NO: 3 - protein, 9 aa, Artificial Sequence
  VH-CDR3 for mouse, chimeric and humanized D2H3, defined by IMGT numbering scheme
TRNRYGLDY

SEQ ID NO: 4 - protein, 8 aa, Artificial Sequence
  VH-CDR1 for mouse, chimeric and humanized D2A4, defined by IMGT numbering scheme
GFTFSSYT

SEQ ID NO: 5 - protein, 8 aa, Artificial Sequence
  VH-CDR2 for mouse, chimeric and humanized D2A4, defined by IMGT numbering scheme
ISGGGSNT

SEQ ID NO: 6 - protein, 14 aa, Artificial Sequence
  VH-CDR3 for mouse, chimeric and humanized D2A4, defined by IMGT numbering scheme
ARQAFYSNYWYFDV

SEQ ID NO: 7 - protein, 15 aa, Artificial Sequence
  VL-CDR1 for mouse, chimeric and humanized D2H3, defined by Chothia or Kabat numbering scheme
RASESVSLHGTRLMH

SEQ ID NO: 8 - protein, 7 aa, Artificial Sequence
  VL-CDR2 for mouse, chimeric and humanized D2H3, defined by Chothia or Kabat numbering scheme
LGSNLES

SEQ ID NO: 9 - protein, 9 aa, Artificial Sequence
  VL -CDR3 for mouse, chimeric and humanized D2H3, defined by IMGT numbering scheme, Chothia numbering scheme or Kabat numbering scheme
QQSIEDPWT

SEQ ID NO: 10 - protein, 11 aa, Artificial Sequence
  VL-CDR1 for mouse, chimeric and humanized D2A4, defined by Chothia or Kabat numbering scheme
LASQTIGTWLA

SEQ ID NO: 11 - protein, 7 aa, Artificial Sequence
  VL -CDR2 for mouse, chimeric and humanized D2A4, defined by Chothia or Kabat numbering scheme
AATSLAD

SEQ ID NO: 12 - protein, 9 aa, Artificial Sequence
  VL -CDR3 for mouse, chimeric and humanized D2A4, defined by IMGT numbering scheme, Chothia numbering scheme or Kabat numbering scheme
QQVSSIPWT

SEQ ID NO: 13 - protein, 116 aa, Artificial Sequence
  VH for mouse D2H3
EVQLQQSGTVLARPGASVKMSCKSSGYTFTNYWMHWVKQRPGQGLEWIGAIFPRNSETNYNQKFKAKAKLTAVTSASTAYMEVSSLTSEDSAVYFCTRNRYGLDYWGQGTSVTVSS

SEQ ID NO: 14 - protein, 116 aa, Artificial Sequence
  VH for huD2H3-V1, huD2H3-V8- huD2H3-V11 and huD2H3-V13
EVQLVQSGAEVKKPGSSVKVSCKSSGYTFTNYWMHWVRQAPGQGLEWIGAIFPRNSETNYNQKFKARATLTADTSTSTAYMELSSLRSEDTAVYFCTRNRYGLDYWGQGTLVTVSS

SEQ ID NO: 15 - protein, 116 aa, Artificial Sequence
  VH for huD2H3-V2
EVQLVQSGAEVKKPGSSVKVSCKASGYTFTNYWMHWVRQAPGQGLEWIGAIFPRNSETNYNQKFKARATLTADTSTSTAYMELSSLRSEDTAVYFCTRNRYGLDYWGQGTLVTVSS

SEQ ID NO: 16 - protein, 116 aa, Artificial Sequence
  VH for huD2H3-V3
EVQLVQSGAEVKKPGSSVKVSCKSSGYTFTNYWMHWVRQAPGQGLEWMGAIFPRNSETNYNQKFKARATLTADTSTSTAYMELSSLRSEDTAVYFCTRNRYGLDYWGQGTLVTVSS

SEQ ID NO: 17 - protein, 116 aa, Artificial Sequence
  VH for huD2H3-V4
EVQLVQSGAEVKKPGSSVKVSCKSSGYTFTNYWMHWVRQAPGQGLEWIGAIFPRNSETNYNQKFKARVTLTADTSTSTAYMELSSLRSEDTAVYFCTRNRYGLDYWGQGTLVTVSS

SEQ ID NO: 18 - protein, 116 aa, Artificial Sequence
  VH for huD2H3-V5
EVQLVQSGAEVKKPGSSVKVSCKSSGYTFTNYWMHWVRQAPGQGLEWIGAIFPRNSETNYNQKFKARATITADTSTSTAYMELSSLRSEDTAVYFCTRNRYGLDYWGQGTLVTVSS

SEQ ID NO: 19 - protein, 116 aa, Artificial Sequence
  VH for huD2H3-V6
EVQLVQSGAEVKKPGSSVKVSCKSSGYTFTNYWMHWVRQAPGQGLEWIGAIFPRNSETNYNQKFKARATLTADESTSTAYMELSSLRSEDTAVYFCTRNRYGLDYWGQGTLVTVSS

SEQ ID NO: 20 - protein, 116 aa, Artificial Sequence
  VH for huD2H3-V7
EVQLVQSGAEVKKPGSSVKVSCKSSGYTFTNYWMHWVRQAPGQGLEWIGAIFPRNSETNYNQKFKARATLTADTSTSTAYMELSSLRSEDTAVYYCTRNRYGLDYWGQGTLVTVSS

SEQ ID NO: 21 - protein, 116 aa, Artificial Sequence
  VH for huD2H3-V12 and huD2H3-V14
EVQLVQSGAEVKKPGSSVKVSCKASGYTFTNYWMHWVRQAPGQGLEWMGAIFPRNSETNYNQKFKARVTITADESTSTAYMELSSLRSEDTAVYYCTRNRYGLDYWGQGTLVTVSS

SEQ ID NO: 22 - protein, 121 aa, Artificial Sequence
  VH for mouse D2A4
EVKLVESGGGLVKPGGSLKLSCAASGFTFSSYTMSWVRQTPAKRLEWVATISGGGSNTYYPDSVKGRFTISRDNARNTLYLQMSSLRSEDTAMYYCARQAFYSNYWYFDVWGAGTTVTVSS

SEQ ID NO: 23 - protein, 121 aa, Artificial Sequence
  VH for huD2A4-V1 and huD2A4-V4
EVQLVESGGGLVQPGGSLRLSCAASGFTFSSYTMSWVRQAPGKGLEWVATISGGGSNTYYPDSVKGRFTISRDNSKNTLYLQMNSLRAEDTAVYYCARQAFYSNYWYFDVWGAGTTVTVSS

SEQ ID NO: 24 - protein, 121 aa, Artificial Sequence
  VH for huD2A4-V2
EVQLVESGGGLVQPGGSLRLSCAASGFTFSSYTMSWVRQAPGKGLEWVSTISGGGSNTYYPDSVKGRFTISRDNSKNTLYLQMNSLRAEDTAVYYCARQAFYSNYWYFDVWGAGTTVTVSS

SEQ ID NO: 25 - protein, 121 aa, Artificial Sequence
  VH for huD2A4-V3
EVQLVESGGGLVQPGGSLRLSCAASGFTFSSYTMSWVRQAPGKGLEWVATISGGGSNTYYPDSVKGRFTISRDNSKNTLYLQMNSLRAEDTAVYYCARQAFYSNYWYFDVWGKGTTVTVSS

SEQ ID NO: 26 - protein, 121 aa, Artificial Sequence
  VH for huD2A4-V5 and huD2A4-V6
EVQLVESGGGLVQPGGSLRLSCAASGFTFSSYTMSWVRQAPGKGLEWVSTISGGGSNTYYPDSVKGRFTISRDNSKNTLYLQMNSLRAEDTAVYYCARQAFYSNYWYFDVWGKGTTVTVSS

SEQ ID NO: 27 - protein, 111 aa, Artificial Sequence
  VL for mouse D2H3
DIVLTQSPGFLAVSLGQRATISCRASESVSLHGTRLMHWYHQKPGQPPKLLISLGSNLESGVPARFSGSGSETDFTLNIHPVEEEDAATYFCQQSIEDPWTFGGGTKLEIK

SEQ ID NO: 28 - protein, 111 aa, Artificial Sequence
  VL for huD2H3-V1 - huD2H3-V7 and huD2H3-V12
DIVLTQSPDSLAVSLGERATINCRASESVSLHGTRLMHWYQQKPGQPPKLLISLGSNLESGVPDRFSGSGSETDFTLTISSLQAEDVAVYFCQQSIEDPWTFGQGTKLEIK

SEQ ID NO: 29 - protein, 111 aa, Artificial Sequence
  VL for huD2H3-V8
DIVMTQSPDSLAVSLGERATINCRASESVSLHGTRLMHWYQQKPGQPPKLLISLGSNLESGVPDRFSGSGSETDFTLTISSLQAEDVAVYFCQQSIEDPWTFGQGTKLEIK

SEQ ID NO: 30 - protein, 111 aa, Artificial Sequence
  VL for huD2H3-V9
DIVLTQSPDSLAVSLGERATINCRASESVSLHGTRLMHWYQQKPGQPPKLLIYLGSNLESGVPDRFSGSGSETDFTLTISSLQAEDVAVYFCQQSIEDPWTFGQGTKLEIK

SEQ ID NO: 31 - protein, 111 aa, Artificial Sequence
  VL for huD2H3-V10
DIVLTQSPDSLAVSLGERATINCRASESVSLHGTRLMHWYQQKPGQPPKLLISLGSNLESGVPDRFSGSGSGTDFTLTISSLQAEDVAVYFCQQSIEDPWTFGQGTKLEIK

SEQ ID NO: 32 - protein, 111 aa, Artificial Sequence
  VL for huD2H3-V11
DIVLTQSPDSLAVSLGERATINCRASESVSLHGTRLMHWYQQKPGQPPKLLISLGSNLESGVPDRFSGSGSETDFTLTISSLQAEDVAVYYCQQSIEDPWTFGQGTKLEIK

SEQ ID NO: 33 - protein, 111 aa, Artificial Sequence
  VL for huD2H3-V13 and huD2H3-V14
DIVMTQSPDSLAVSLGERATINCRASESVSLHGTRLMHWYQQKPGQPPKLLIYLGSNLESGVPDRFSGSGSGTDFTLTISSLQAEDVAVYYCQQSIEDPWTFGQGTKLEIK

SEQ ID NO: 34 - protein, 107 aa, Artificial Sequence
  VL for mouse D2A4
DIQMTQSPASQSASLGESVTITCLASQTIGTWLAWYQQTPGKSPQLLIYAATSLADGVPSRFSGSGSGTKFSFKISSLQAEDFASYYCQQVSSIPWTFGGGTKLEIR

SEQ ID NO: 35 - protein, 107 aa, Artificial Sequence
  VL for huD2A4-V1 - huD2A4-V3 and huD2A4-V5
DIQMTQSPSSVSASVGDRVTITCLASQTIGTWLAWYQQKPGKSPKLLIYAATSLADGVPSRFSGSGSGTDFTLTISSLQPEDFATYYCQQVSSIPWTFGGGTKVEIK

SEQ ID NO: 36 - protein, 107 aa, Artificial Sequence
  VL for huD2A4-V4 and huD2A4-V6
DIQMTQSPSSVSASVGDRVTITCLASQTIGTWLAWYQQKPGKAPKLLIYAATSLADGVPSRFSGSGSGTDFTLTISSLQPEDFATYYCQQVSSIPWTFGGGTKVEIK

SEQ ID NO: 37 - protein, 7 aa, Artificial Sequence
  VH-CDR1 for mouse, chimeric and humanized D2H3, defined by Chothia numbering scheme
GYTFTNY

SEQ ID NO: 38 - protein, 5 aa, Artificial Sequence
  VH-CDR1 for mouse, chimeric and humanized D2H3, defined by Kabat numbering scheme
NYWMH

SEQ ID NO: 39 - protein, 6 aa, Artificial Sequence
  VH-CDR2 for mouse, chimeric and humanized D2H3, defined by Chothia numbering scheme
FPRNSE

SEQ ID NO: 40 - protein, 17 aa, Artificial Sequence
  VH-CDR2 for mouse, chimeric and humanized D2H3, defined by Kabat numbering scheme
AIFPRNSETNYNQKFKA

SEQ ID NO: 41 - protein, 7 aa, Artificial Sequence
  VH-CDR3 for mouse, chimeric and humanized D2H3, defined by Chothia or Kabat numbering scheme
NRYGLDY

SEQ ID NO: 42 - protein, 10 aa, Artificial Sequence
  VL-CDR1 for mouse, chimeric and humanized D2H3, defined by IMGT numbering scheme
ESVSLHGTRL

SEQ ID NO: 43 - protein, 3 aa, Artificial Sequence
  VL-CDR2 for mouse, chimeric and humanized D2H3, defined by IMGT numbering scheme
LGS

SEQ ID NO: 44 - protein, 7 aa, Artificial Sequence
  VH-CDR1 for mouse, chimeric and humanized D2A4, defined by Chothia numbering scheme
GFTFSSY

SEQ ID NO: 45 - protein, 5 aa, Artificial Sequence
  VH-CDR1 for mouse, chimeric and humanized D2A4, defined by Kabat numbering scheme
SYTMS

SEQ ID NO: 46 - protein, 6 aa, Artificial Sequence
  VH-CDR2 for mouse, chimeric and humanized D2A4, defined by Chothia numbering scheme
SGGGSN

SEQ ID NO: 47 - protein, 17 aa, Artificial Sequence
  VH-CDR2 for mouse, chimeric and humanized D2A4, defined by Kabat numbering scheme
TISGGGSNTYYPDSVKG

SEQ ID NO: 48 - protein, 12 aa, Artificial Sequence
  VH-CDR3 for mouse, chimeric and humanized D2A4, defined by Chothia or Kabat numbering scheme
QAFYSNYWYFDV

SEQ ID NO: 49 - protein, 6 aa, Artificial Sequence
  VL-CDR1 for mouse, chimeric and humanized D2A4, defined by IMGT numbering scheme
QTIGTW

SEQ ID NO: 50 - protein, 3 aa, Artificial Sequence
  VL -CDR2 for mouse, chimeric and humanized D2A4, defined by IMGT numbering scheme
AAT

SEQ ID NO: 51 - protein, 330 aa, Artificial Sequence
  human IgG1 heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 107 aa, Artificial Sequence [sequence omitted]

SEQ ID NO: 53 - protein, 371 aa, Artificial Sequence
  human PD-1-Fc
LDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSL

SEQ ID NO: 54 - protein, 453 aa, Artificial Sequence
  human PD-L1-Fc
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - DNA, 348 bp, Artificial Sequence
  VH for mouse D2H3
gaggttcagctccagcagtctgggactgtgctggcaaggcctggggcctcagtgaagatgtcctgcaagtcttctggctacacctttaccaactactggatgcactgggtaaaacagaggcctggacagggtctggaatggattggcgctatttttcctagaaatagtgagactaactacaaccagaaatttaaggccaaggccaaactgactgcagtcacatctgccagcactgcctacatggaggtcagcagcctgacaagtgaggactctgcggtctatttctgtacgaggaataggtatggtctggactactggggtcaaggaacctcagtcaccgtctcctca

SEQ ID NO: 56 - DNA, 348 bp, Artificial Sequence
  VH for huD2H3-V12 and huD2H3-V14
gaggtgcagctggtgcagagcggcgccgaggtgaagaagcccggctccagcgtgaaggtgagctgcaaggccagcgggtacaccttcacaaactactggatgcactgggtgaggcaggccccagggcagggcctggagtggatgggcgccatcttccccaggaacagcgagacaaactacaaccagaagttcaaggccagggtgacaatcacagccgatgagagcaccagcacagcctacatggagctgagcagcctgcggagcgaggacaccgccgtgtactactgcacccggaaccggtacgggctggattactgggggcaggggaccctggtgacagtgagcagc

SEQ ID NO: 57 - DNA, 363 bp, Artificial Sequence
  VH for mouse D2A4
gaagtgaagctggtggagtctgggggaggcttagtgaagcctggagggtccctgaaactctcctgtgcagcctctggattcactttcagtagctataccatgtcttgggttcgccagactccggcgaagaggctggagtgggtcgcaaccattagtggaggtggtagtaacacctactatcctgacagtgtgaagggccgattcaccatctccagagacaatgccaggaacaccctgtacctgcaaatgagcagtctgaggtctgaggacacggccatgtattactgtgcaagacaagccttctatagtaactactggtacttcgacgtctggggcgcagggaccacggtcaccgtctcctca

SEQ ID NO: 58 - DNA, 363 bp, Artificial Sequence
  VH for huD2A4-V5 and huD2A4-V6
gaggtgcagctggtggagagcggggggggactggtgcagccaggaggaagcctgagactgagctgtgccgcaagcgggttcacatttagtagttacacaatgagctgggtgagacaggcccccggaaaaggactggagtgggtgtctactatttcaggagggggaagcaacacctattatcccgatagtgtgaagggcagattcacaatcagtagagacaacagcaagaacaccctgtacctgcagatgaacagcctgagagccgaggacaccgccgtgtactactgcgccagacaggccttttacagtaactattggtacttcgacgtgtggggaaagggaaccacagtgactgtgagcagc

SEQ ID NO: 59 - DNA, 333 bp, Artificial Sequence
  VL for mouse D2H3
gacatcgtgctgacccaatctccaggttttttggctgtgtctctagggcagagggccaccatctcctgcagagccagtgaaagtgtcagtcttcatggtactcgtttaatgcactggtaccatcagaaaccaggacagccacccaaactcctcatctctcttggatccaacctagagtctggagtccctgccaggttcagtggcagtgggtctgagacagacttcaccctcaacatccatcctgtggaggaggaggatgctgcaacctatttctgtcagcaaagtattgaggatccgtggacgttcggtggaggcaccaagctggaaatcaaa

SEQ ID NO: 60 - DNA, 333 bp, Artificial Sequence
  VL for huD2H3-V13 and huD2H3-V14
gatatcgtgatgacacagagccccgatagcctggccgtgagcctgggggagcgggccaccatcaactgccgggccagcgagagcgtgagcctgcacgggacaaggctgatgcactggtaccagcagaagcccggccagccccccaagctgctgatctacctggggagcaacctggagagcggggtgcccgataggttcagcggcagcgggagcggcacagatttcacactgaccatcagcagcctgcaggccgaggacgtggccgtgtactactgccagcagagcatcgaggacccctggaccttcggccagggcacaaagctggagatcaag

SEQ ID NO: 61 - DNA, 321 bp, Artificial Sequence
  VL for mouse D2A4
gacattcagatgacccagtctcctgcctcccagtctgcatctctgggagaaagtgtcaccatcacatgcctggcaagtcagaccattggtacatggttagcatggtatcagcagacaccagggaaatctcctcagctcctgatttatgctgcaaccagcttggcagatggggtcccatcaaggttcagtggtagtggatctggcacaaagttttctttcaagatcagcagcctacaggctgaagattttgcaagttattactgtcaacaagtttccagtattccgtggacgttcggtggaggcaccaagctggaaatcaga

SEQ ID NO: 62 - DNA, 321 bp, Artificial Sequence
  VL for huD2A4-V4 and huD2A4-V6
gacattcagatgacccagagccccagcagcgtgagcgccagcgtgggagacagagtgaccataacctgcctggccagccaaaccataggcacctggctggcctggtaccagcagaaacccggcaaagcccccaaactgctcatctacgccgccaccagcctggctgacggagtgccaagcagattctccggtagcggcagcggcaccgacttcaccctgactatcagcagcctccaacccgaagacttcgccacctactactgccaacaggtctcctccattccctggaccttcggaggcggcaccaaggtggagatcaaa

SEQ ID NO: 63 - DNA, 990 bp, Artificial Sequence
  human IgG1 heavy chain constant region
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 64 - DNA, 321 bp, Artificial Sequence
  human kappa light chain constant region
cgtacggtggcggcgccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 65 - protein, 324 aa, Artificial Sequence
  Heavy chain constant region for mouse antibodies
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 66 - protein, 107 aa, Artificial Sequence
  Light chain constant region for mouse antibodies
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRGEC

SEQ ID NO: 67 - DNA, 972 bp, Artificial Sequence
  Heavy chain constant region for mouse antibodies
gccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 68 - DNA, 321 bp, Artificial Sequence
  Light chain constant region for mouse antibodies
cgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgactaaggacgagtatgaacgacataacagctatacctgtgaggccactcacaagacatcaacttcacccattgtcaagagcttcaacaggggagagtgt